(19) United States
(12) Patent Application Publication
(10) Pub. No.: US 2004/0153249 A1
(43) Pub. Date: Aug. 5, 2004

(54) System, software and methods for biomarker identification

(75) Inventors: Zhen Zhang, Dayton, MD (US); Daniel Wanyui Chan, Clarksville, MD (US)

Correspondence Address: EDWARDS & ANGELL, LLP, P.O. BOX 55874, BOSTON, MA, 02205, US

(73) Assignee: The Johns Hopkins University, Baltimore, MD

(21) Appl. No.: 10/635,241

(22) Filed: Aug. 5, 2003

Related U.S. Application Data
(60) Provisional application No. 60/401,837, filed on Aug. 6, 2002.
(60) Provisional application No. 60/441,727, filed on Jan. 21, 2003.
(60) Provisional application No. 60/460,342, filed on Apr. 4, 2003.
(60) Provisional application No. 60/464,757, filed on Apr. 22, 2003.

(51) Int. Cl. G06F 15/18; G06F 19/00; G01N 33/48; G01N 33/50

(52) U.S. Cl. 702/19; 706/1

(57) ABSTRACT

The invention provides a method, system and software to screen for, identify and validate biomarkers that are predictive of a biological state, such as a cell state and/or patient status.

### RELATED APPLICATIONS

This application claims the benefit of U.S. provisional application No. 60/401,837, filed Aug. 6, 2002; U.S. provisional application No. 60/441,727, filed Jan. 21, 2003; U.S. provisional application No. 60/460,342, filed Apr. 4, 2003; and U.S. provisional application No. 60/464,757, filed Apr. 22,2003, all of which applications are incorporated by reference herein in their entirety.

### FIELD OF THE INVENTION

The invention relates to systems, software and methods for identifying biomarkers.

### BACKGROUND

Genomic and proteome analysis supplies a wealth of information regarding the numbers and forms of proteins expressed in a cell and provides the potential to identify for each cell, a profile of expressed proteins characteristic of a particular cell state. In some cases, this cell state may be characteristic of an abnormal physiological response associated with a disease. Consequently, identifying and comparing a cell state from a patient with a disease to that of a corresponding cell from a normal patient can provide opportunities to diagnose and control treatment of disease.

Recent advances in transcriptional and proteomic profiling technology have made it possible to apply computational methods to detect changes in expression patterns and their association to disease conditions, thereby hastening the identification of novel markers that may contribute to multi-marker combinations with highly accurate diagnostic performance.

While high throughput screening methods provide large data sets of gene expression information, the challenge of bioinformatics remains to develop robust methods for organizing the data into patterns that are reproducibly diagnostic for diverse populations of individuals. The commonly accepted approach has been to pool data from multiple sources to form a combined data set and then to divide the data set into a discovery/training set and a test/validation set. However, both transcription profiling data and protein expression profiling data are often characterized by a large number of variables relative to the available number of samples.

Observed differences between expression profiles of specimens from groups of patients or controls are typically overshadowed by (1) biological variability or unknown sub-phenotypes within the disease or control populations; (2) site-specific biases due to difference in study protocols, specimens handling, etc.; (3) biases due to differences in instrument conditions (e.g., chip batches, etc); and/or (4) variations due to measurement error. False discovery of drug targets remains a serious issue, especially considering the cost and effort typically required for “post-discovery” work such as protein/gene identification and further validation for potential biomarkers.

### SUMMARY OF THE INVENTION

Systematic biases due to site-specific factors can only be detected through careful analysis and comparison of data from multiple sources. The invention provides systems, software and methods for analyzing expression profiling data from multiple sources (e.g., such as clinical trial sites) to overcome the possible systematic biases in expression data typically generated in such analyses, thereby reducing the probability of false discovery of drug targets. In one preferred aspect, the invention combines the use bioinformatics and expression profiling of specimens from multiple sources to screen for, identify, and validate biomarkers for a particular biological state or condition of interest. The measurement of these markers in patient samples can provide information that may be the presence, absence or severity of a condition or characteristic of a patient such as a human being. In one aspect, the condition or characteristic is the presence, predisposition or risk of recurrence of a disease.

The invention provides bioinformatics tools to analyze expression profiling data of samples from two or more independent sources in a way which reduces the sources of variability and biases which result in identification of false targets during the drug discovery process. In contrast to prior art methods, data from multiple sources are NOT pooled together into a combined data set and then divided into a discovery/training set and a test/validation set. Instead, data from multiple sources (e.g., such as multiple different clinical trial sites) are analyzed separately and independently from the others. For each source, sufficient sample size and statistical re-sampling methods (e.g., such as bootstrap analysis) help to discover biomarkers that perform well in a representative population and perform consistently well among different randomly selected subpopulations. The use of a re-sampling procedure reduces the compound impact of biological variability and large number of variables in gene expression profiling data.

Further, the use of replicates of samples helps to alleviate problems associated with possible measure errors and limitations in instrument precision.

The invention involves developing at least two different learning sets (discovery data sets) that have been developed independently of each other. Each learning set includes subject data (data points) from a plurality of subjects. The subject data from each subject indicates a phenotype (form of a biological state class or pathology status) to which the subject belongs, and each subject is classified into one of a plurality of different pathology classes. The different phenotypes generally are pathology related, for example, diseased v. normal, different disease stages, etc. However, they can include any measurable biological characteristic. Each learning set has subject data from at least two subjects belonging to each of the phenotypes. The subject data from each subject comprises measurements of a plurality of data elements from each subject sample.

The results from the separately and independently conducted analyses are then cross-compared to identify a subset of potential biomarkers that share a comparable level of performance on data from each individual source AND share the same up/down regulation patterns between the different groups of samples across the multiple sources of data.

Biomarkers selected from the cross-comparison are then used to develop a multivariate classification model that classifies a sample into one of the biological state classes or conditions.

This subset of potential biomarkers, preferably, will be further validated using another independent validation data set. Furthermore, the identities of these potential biomarkers, preferably, will be identified and their performance validated using additional samples and with additional methods (e.g., including, but not limited to immunoassays).

In one preferred aspect, the expression profiling data evaluated is proteomic profiling data (i.e., data relating to the expression of proteins and their modified and processed forms). For example, the method is particularly amenable for use with mass spectrometry-based analysis of a proteome. Therefore, in one aspect, the method is used to screen for, identify, and validate biomarkers characterized by molecular weight and/or by their known protein identities. The markers can be resolved from other proteins in a sample by using a variety of fractionation techniques, e.g., chromatographic separation coupled with mass spectrometry, or by traditional immunoassays. Mass spectral data obtained from independently evaluated data sets are evaluated using a learning technique (which may be supervised or unsupervised) to identify biomarkers or sets of biomarkers with desired confidence levels (i.e., discriminatory power). Data (e.g., types of biomarkers expressed, level of expression for each biomarker) from independent data sets are cross-compared to identify those markers diagnostic of one or more characteristics of the data sets. Such characteristics can include the presence of a condition shared by members of the data sets, such as the presence of a disease.

In a preferred aspect, data is obtained by SELDI analysis of cellular protein samples and data obtained relating to samples within each data set relates to the mass-to-charge ratios or molecular weights of biomolecules (e.g., such as peptides) present in samples from patients belonging to the data set.

The expression profile (e.g., presence, absence, quantity) of the biomarkers in a sample can be used to identify the status of cell, a tissue, organ, and/or patient. In certain aspects, the expression profile of a single biomarker is indicative of the status. In other aspects, the expression profile of a plurality of markers is indicative of the status. In a particularly preferred embodiment, SELDI (Surface-Enhanced Laser-Desorption and Ionization) mass spectrometry is performed.

Accordingly, the invention provides, a method comprising:

(a) providing at least a first and a second independent discovery data set wherein:

(i) the data sets comprise a plurality of biological state classes;

(ii) each data set comprises a plurality of data points, wherein each data point exhibits one form of a biological state class and each data set comprises a plurality of data points belonging to each of the classes;

(iii) each data point comprises a plurality of data elements, each data element characterized by a value, wherein all data points share a plurality of common data elements; and

(b) qualifying each common data element, independently for each dataset, based on the ability of the data element to classify a data point into a form of biological state class, as a function of data element value;

(c) selecting an initial subset of data elements within each data set, and

(d) selecting an intersection subset of data elements from the initial subsets, wherein each data element in the intersection subset is a member of a majority of the initial subsets.

In one aspect, the step of selecting the initial subsets comprises using the discovery data sets to train a learning algorithm wherein the learning algorithm ranks the data elements based on a quantitative measure of ability to classify. The learning algorithm used may be supervised or unsupervised.

In one aspect, the training method is a supervised method such as support vector machine analysis. In another aspect, a statistical method such as linear discrimination analysis is used. Further, the two approaches can be combined. In a preferred method, a unified maximum separability analysis (UMSA) method is used. This is particularly advantageous, when the number of data points in a data set is small.

In a further aspect, data elements in each data set are independently re-sampled before cross-comparison.

The methods may further comprise selecting candidate biomarkers from the selected data elements and testing one or more of the candidate biomarkers on a validation data set.

In one aspect, the biological state class is a cell state. In another aspect, the biological state class is a patient status.

In a further aspect, biological state class represents the presence of a disease; absence of a disease; progression of a disease; risk for a disease; stage of disease; likelihood of recurrence of disease; a genotype; a phenotype; exposure to an agent or condition; a demographic characteristic; resistance to agent, and sensitivity to an agent. The genotype may be an HLA haplotype; a mutation in a gene; a modification of a gene, and combinations thereof. The agent may include, but is not limited to a toxic substance, a potentially toxic substance, an environmental pollutant, a candidate drug, and a known drug. The demographic characteristic may include, but is not limited to: age, gender, weight; family history; and history of preexisting conditions. Sensitivity to an agent may include responsiveness to a drug.

In one aspect, one or more candidate biomarkers is/are diagnostic of the presence of a disease, risk of developing a disease, risk of recurrence of a disease, or stage of the disease. In another aspect, values of the data elements in a data point represent levels and/or frequency of components in a data point sample. Exemplary components include but are not limited to nucleic acids, proteins, polypeptides, peptides, carbohydrates and modified or processed forms thereof. In one aspect, levels of components are measured in by an expression profiling assay. In another aspect, the expression profiling assay comprises measuring the amount and/or form of a nucleic acid (e.g., such as RNA). In a still another aspect, expression profiling may also include measuring amplification, mutation, or modification of DNA. In a further aspect, the expression profiling assay comprises measuring the amount and/or form of a protein, polypeptide or peptide, such as by mass spectrometry (e.g., SELDI). In still a further aspect, the expression profiling assay comprises measuring the amount and/or form of a carbohydrate.

In one aspect, data elements of data points comprise data relating to the cellular localization of components in a sample.

In another aspect, expression profiling comprises contacting samples with substrate comprising binding partners for specifically binding to sample components having selected characteristics and identifying sample components bound to the substrate. Suitable binding partners include, but are not limited to: cationic molecules; anionic molecules; metal chelates; antibodies; single- or double-stranded nucleic acids; proteins, peptides, amino acids; carbohydrates; lipopolysaccharides; sugar amino acid hybrids; molecules from phage display libraries; biotin; avidin; streptavidin; and combinations thereof. In one aspect, the binding partners are arrayed on the substrate.

In still another aspect, an assay used to measure levels of data elements in training data sets from which candidate biomarkers are identified is different from an assay used to measure data elements in a validation data set used to validate the candidate biomarker.

In one aspect, the assay used to measure levels of data elements in training data sets is SELDI.

In another aspect, the assay used to measure levels of data elements in validation data sets is an immunoassay.

In a further aspect, the assay used to measure levels of data elements in training data sets is SELDI and the assay used to measure levels of data elements in validation data sets is an immunoassay.

Independently collected data sets may collected from different locations, using different collection protocols, and/or are collected from different populations. In one aspect, each data set of the plurality of data sets is from a different clinical trial site.

In one aspect, there are at least about 100 data points per data set.

In another aspect, there are at least about 50 data elements per data point.

The invention further provides a computer program product comprising a computer readable medium having computer readable program code embodied in the medium for causing an application program to execute on a computer with a database; the program product comprising:

a. a first computer readable program code providing instructions for causing a computer to input data representing values of a plurality of data elements, the plurality of data elements from data points representing a plurality of independently collected discovery data sets, each data element characterized by a value, wherein all data points share a plurality of common data elements;

b. a second computer readable program code providing instructions for qualifying each common data element, independently for each data set, based on the ability of the data element to classify a data point into a biological state class, as a function of data element value and for selecting an initial subset of data elements within each data set, and

c. a third computer readable program code providing instructions for selecting an intersection subset of data elements from the initial subsets, wherein each data element in the intersection subset is a member of a majority of the initial subsets.

In one aspect, the program product comprises a fourth computer readable program code for selecting candidate biomarkers from the ranked data elements and testing one or more of the candidate biomarkers on a validation data set.

In another aspect, the biological state class is a cell state. In a further aspect, the biological state is a patient status. Generally, data points represent biological samples having the at least one characteristic of the biological state. The characteristic may be presence of a disease; absence of a disease; progression of a disease; risk for a disease; stage of disease; likelihood of recurrence of disease; a genotype; a phenotype; exposure to an agent or condition; a demographic characteristic; resistance to agent, and sensitivity to an agent (e.g., responsiveness to a drug). The genotype may be selected from the group consisting of an HLA haplotype; a mutation in a gene; a modification of a gene, and combinations thereof. In one aspect, the agent is selected from the group consisting of a toxic substance, a potentially toxic substance, an environmental pollutant, a candidate drug, and a known drug. The demographic characteristic may be one or more of age, gender, weight; family history; and history of preexisting conditions.

In another aspect, one or more candidate biomarkers is/are diagnostic of the presence of a disease, risk of developing a disease, risk of recurrence of a disease, or stage of the disease.

In a further aspect, values of the data elements in a data point represent levels and/or frequency of components in a data point sample, e.g., such as nucleic acids, proteins, polypeptides, peptides, carbohydrates and modified or processed forms thereof. In one aspect, levels are measured in an expression profiling assay. For example, in one aspect, the expression profiling assay comprises measuring the amount and/or form of a nucleic acid (e.g., such as RNA, or an amplified, mutated and/or modified form of DNA).

In another aspect, the expression profiling assay comprises measuring the amount and/or form of a protein, polypeptide or peptide, such as by mass spectrometry (e.g., SELDI).

In still another aspect, the expression profiling assay comprises measuring the amount and/or form of a carbohydrate. In a further aspect, data elements of data points comprise data relating to the cellular localization of components (e.g., mRNA, proteins) in a sample.

In one aspect, expression profiling comprises contacting samples with substrate comprising binding partners for specifically binding to sample components having selected characteristics and identifying sample components bound to the substrate. Suitable binding partners include but are not limited to cationic molecules; anionic molecules; metal chelates; antibodies; single- or double-stranded nucleic acids; proteins, peptides, amino acids; carbohydrates; lipopolysaccharides; sugar amino acid hybrids; molecules from phage display libraries; biotin; avidin; streptavidin; and combinations thereof. In one preferred aspect, binding partners are arrayed on the substrate.

The computer readable program product may additionally comprise a program code for independently re-sampled data elements in each data set before cross-comparison. Selecting data elements may be done using a learning technique. The learning technique may be supervised or unsupervised. In one aspect, the supervised learning technique comprises support vector machine analysis. In another aspect, the supervised learning technique comprises performing a statistical method, such as linear discrimination analysis. In a further aspect, the two methods are combined. In one preferred aspect, when the number of data points is small, the learning technique comprises performing UMSA.

The assay used to measure levels of data elements in training data sets from which candidate biomarkers are identified may be different from an assay used to measure data elements in a validation data set used to validate the candidate biomarker. In one aspect, the assay used to measure levels of data elements in training data sets is SELDI. In another aspect, the assay used to measure levels of data elements in validation data sets is an immunoassay. In a further aspect, the assay used to measure levels of data elements in training data sets is SELDI and the assay used to measure levels of data elements in validation data sets is an immunoassay.

In one aspect, each data set evaluated by the computer program product is from a different clinical trial site. In another aspect, independently collected data sets are collected from different locations, using different collection protocols, and/or are collected from different populations.

In one aspect, there are at least about 100 data points per data set. In another aspect, there are at least about 50 data elements per data point.

The invention also provides a system comprising:

(a) one or more processors for:

(i) receiving input data representing values of a plurality of data elements, the plurality of data elements from data points representing a plurality of independently collected discovery data sets, each data element characterized by a value, wherein all data points share a plurality of common data elements;

(ii) executing computer readable program code providing instructions for qualifying each common data element, independently for each data set, based on the ability of the data element to classify a data point into a biological state class, as a function of data element value and for selecting an initial subset of data elements within each data set; and

(iii) executing computer readable program code providing instructions for selecting an intersection subset of data elements from the initial subsets, wherein each data element in the intersection subset is a member of a majority of the initial subsets.

In one aspect, the system further comprises one or more devices for providing input data to the one or more processors.

Preferably, the system, further comprises a memory for storing a data set of ranked data elements. In one aspect, a processor for executing further derives training rules from selected data sets to predict the presence of the biological state in a test data point representing a sample being tested for the biological state.

In another aspect, the device for providing input data comprises a detector for detecting the characteristic of the data element, e.g., such as a mass spectrometer or gene chip reader.

In one aspect, the biological state is a cell state. In another aspect, the biological state is a patient status.

In one aspect, data points comprise biological samples having the at least one characteristic of the biological state. In a further aspect, at least one common characteristic is selected from the group consisting of the presence of a disease; absence of a disease; progression of a disease; risk for a disease; stage of disease; likelihood of recurrence of disease; a genotype; a phenotype; exposure to an agent or condition; a demographic characteristic; resistance to agent, and sensitivity to an agent (e.g., responsiveness to a drug). Genotype may include, for example, an HLA haplotype; a mutation in a gene; a modification of a gene, and combinations thereof. Exemplary agents include but are not limited to a toxic substance, a potentially toxic substance, an environmental pollutant, a candidate drug, and a known drug. A demographic characteristic may include, but is not limited to: one or more of age, gender, weight; family history; and history of preexisting conditions.

In one aspect, one or more data elements are candidate biomarkers diagnostic of the presence of a disease, risk of developing a disease, risk of recurrence of a disease, or stage of the disease.

In another aspect, values of the data elements in a data point represent levels and/or frequency of components in a data point sample, e.g., such as nucleic acids, proteins, polypeptides, peptides, carbohydrates and modified or processed forms thereof.

In one aspect, the levels are measured in by an expression profiling assay. The expression profiling assay may comprise, for example, measuring the amount and/or form of a nucleic acid (e.g., such as RNA or an amplified, mutated and/or modified RNA. In another aspect, the expression profiling assay comprises measuring the amount and/or form of a protein, polypeptide or peptide (e.g., by mass spectroscopy or SELDI).

In still another aspect, the expression profiling assay comprises measuring the amount and/or form of a carbohydrate. In a further aspect, data elements of data points comprise data relating to the cellular localization of components in a sample.

In one aspect, expression profiling comprises contacting samples with substrate comprising binding partners for specifically binding to sample components having selected characteristics and identifying sample components bound to the substrate.

Suitable binding partners include but are not limited to cationic molecules; anionic molecules; metal chelates; antibodies; single- or double-stranded nucleic acids; proteins, peptides, amino acids; carbohydrates; lipopolysaccharides; sugar amino acid hybrids; molecules from phage display libraries; biotin; avidin; streptavidin; and combinations thereof. In one preferred aspect, binding partners are arrayed on the substrate.

The system may independently re-sample data elements in each data set before cross-comparison.

In one aspect, biomarker selection is performed using a learning technique, which may be supervised or unsupervised. An exemplary supervised learning technique comprises support vector machine analysis. A statistical method may also be used such as linear discrimination analysis. In some aspects, a combination of the two approaches is used. In one preferred aspect, where sample size is small, biomarker selection is performed by UMSA.

The assay used to measure levels of data elements in training data sets from which candidate biomarkers are identified is different from an assay used to measure data elements in a validation data set used to validate the candidate biomarker. The assay used to measure levels of data elements in the training set may be SELDI. The assay used to measure data elements may be an immunoassay. The assay used to measure data elements in the training set may be SELDI while the assay to measure data elements in the validation data set is an immunoassay. In certain aspects, therefore, more than one device may provide data input to the system.

In one aspect, each data set of the plurality of data sets is from a different clinical trial site. In another aspect, independently collected data sets are collected from different locations, using different collection protocols, and/or are collected from different populations.

In one aspect, there are at least about least about 100 data points per data set. In another aspect, there are at least about 50 data elements per data point per data set.

### BRIEF DESCRIPTION OF THE FIGURES

The objects and features of the invention can be better understood with reference to the following detailed description and accompanying drawings.

FIG. 1A is a schematic diagram of a method according to the invention for screening for, identifying and validating biomarkers. FIG. 1B is a diagram of a study design for identification of ovarian cancer biomarkers implemented using the method shown in FIG. 1A.

FIG. 2 is a snapshot of a user interface and 3-Dimensional (“3D”) plot of a UMSA component module in a system according to one embodiment of the invention.

FIG. 3 is a snapshot of the user interface of the backward stepwise variable selection module according to one embodiment of the present invention.

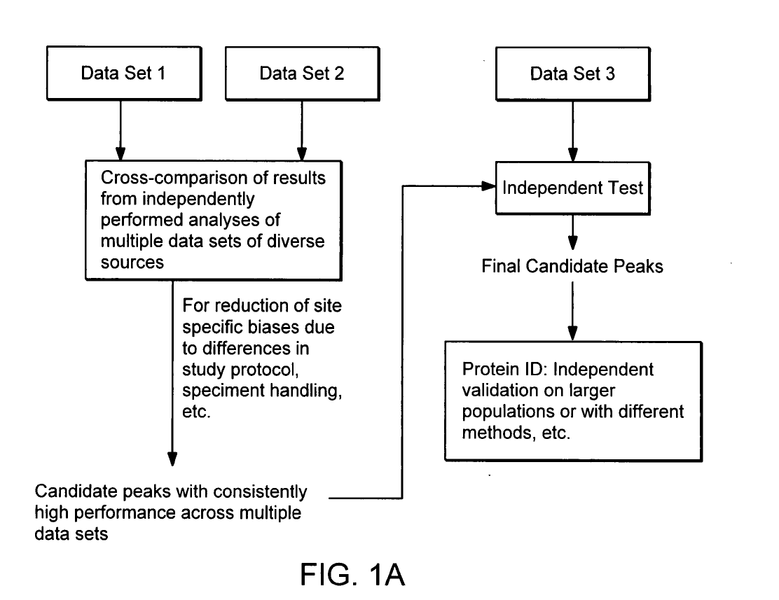

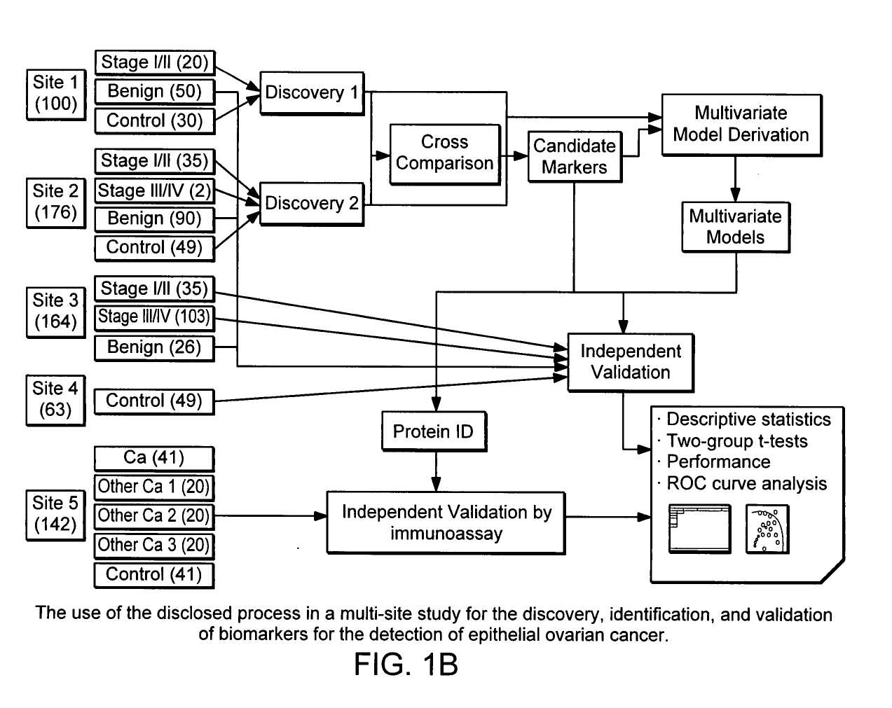

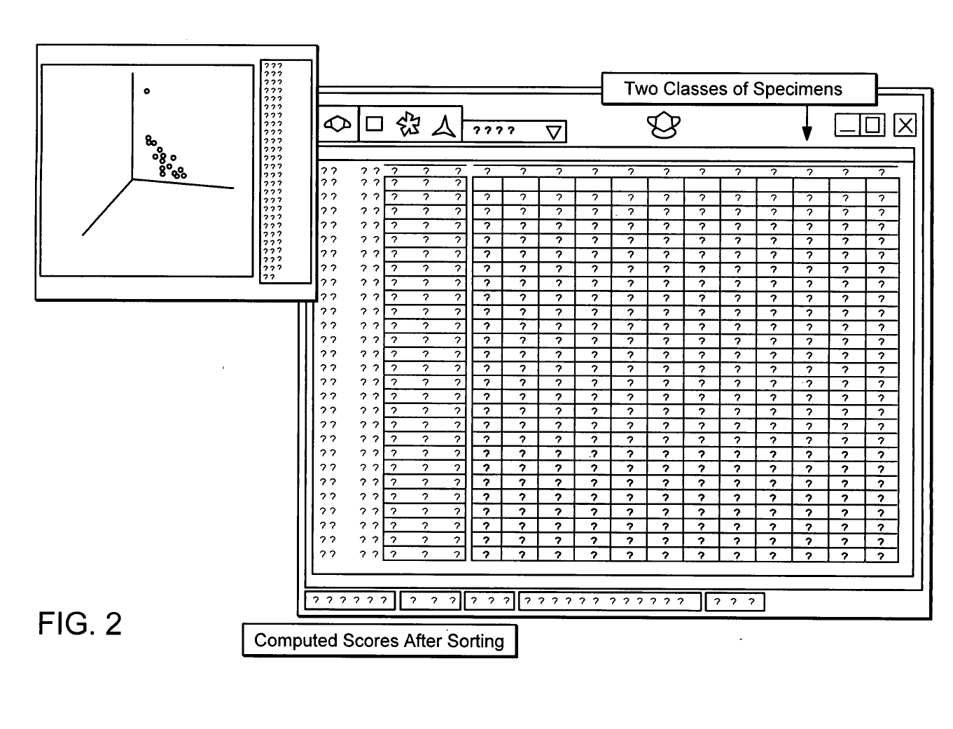

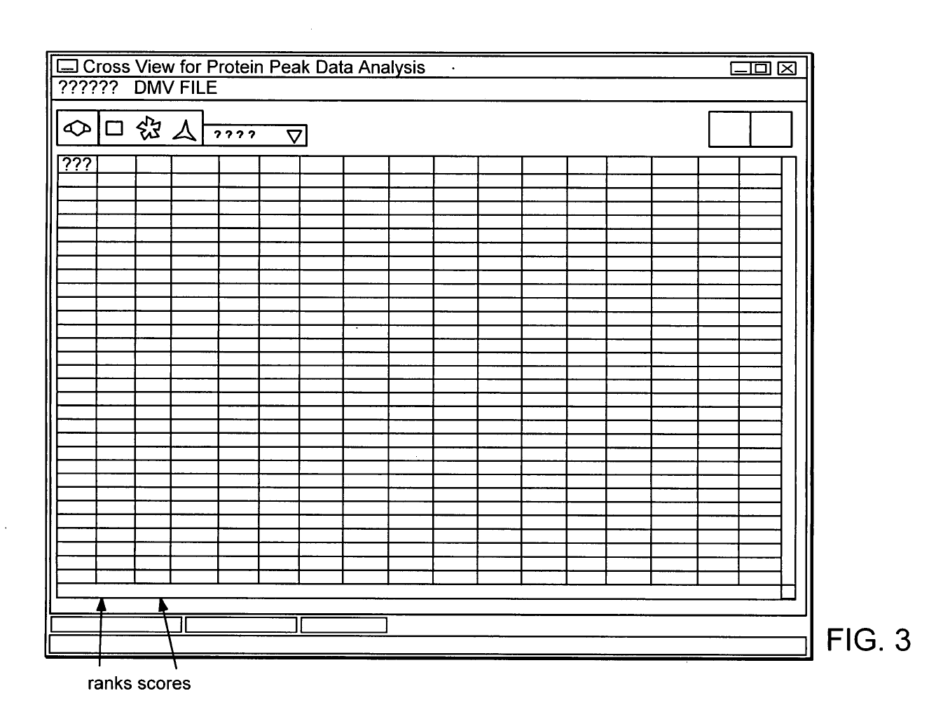

### DETAILED DESCRIPTION

The invention provides a method, system and software to screen for, identify and validate biomarkers which are predictive of a biological state, such as a cell state and/or patient status.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

Definitions

As used herein, the following terms have the meanings ascribed to them unless specified otherwise.

As used in the specification and claims, the singular form “a”, “an” and “the” include plural references unless the context clearly dictates otherwise. For example, the term “a cell” includes a plurality of cells, including mixtures thereof. The term “a protein” includes a plurality of proteins.

Also, as used in the description herein and throughout the claims that follow, the meaning of “in” includes “in” and “on” unless the context clearly dictates otherwise.

A “biomarker” in the context of the present invention refers to a biomolecule, e.g., a protein or a modified, cleaved or fragmented form thereof, a nucleic acid, carbohydrate, metabolite; intermediate, etc. which is differentially present in a sample and whose presence, absence or quantity is indicative of the status of the source of the sample (e.g., cell(s), tissue(s), a patient, etc). The term “biomarker” is used interchangeably with the term “marker.”

“Data set” refers to a set of data whose elements are data points.

“Data point” refers to an element of a dataset, e.g., a subject sample, identified for example, by a label or patient number identifying the source of the sample.

“Biological state class” refers to a biological characteristic into which a data point can be classed. Each dataset comprising data points 1 through i, will have at least two data points representing one of at least two forms of a biological state class. For example the class, present in the sample source providing the data point (class +1) or absent in the sample source providing the data point (class −1). In one aspect, the class −1 data point represents a control (e.g., negative for a disease), though this is not necessarily so. For example, in certain aspects, the class +1 sample represents a certain stage of a disease (e.g., malignant cancer) while class −1 represents another stage of the disease (e.g., benign cells). What the state class represents will be governed by the nature of the diagnostic test the biomarkers are being selected for. Examples of biological state classes are pathology (pathological v. non-pathological (e.g., cancer v. non-cancer)), drug response (drug responder v. drug non-responder), toxic response (toxic response v. non-toxic response), prognosis (progressor to disease state v. non-progressor to disease state), and, most generally, phenotype (phenotypic condition present v. phenotypic condition absent).

“Data element” refers to features of a data point representing characteristics of the data point. For example, in one aspect, data elements represent expression values of a plurality of different genes in a sample. In another aspect, data elements represent peaks detected by mass spectrometry. In another aspect, data elements represent a variety of phenotypic characteristics, e.g., levels of any biologically significant analyte (e.g., clinical chemistry or hematology laboratory panels), responses to questions in an evaluation test, elements of a medical history, etc..

“Data element value” refers to a value assigned to a data element. The value may be qualitative or quantitative, for example “present or absent,” “high. medium or low,” or a measured numerical amount.

“Qualifying” a data element refers to assigning a value to the data element to which a selection criterion can be applied.

“Selection criteria” refers to a criterion or criteria established by a user implementing the method applied to a qualifier to select a data element into an initial subset. The selection criteria may be a cut-off for a numerical qualifier or a class for a qualitiative qualifier. Examples of cut-off criteria are “data elements in the top ten percent of discriminatory power” or “data elements providing at least 80% specificity and at least about 70% sensitivity.” Examples of class criteria are “good” or “bad” data elements based on the qualifier; to some extent this will depend on the nature of the biological state class of interest as for a disease with few diagnostic markers data elements with lower specificity or sensitivity may be selected with a lower numerical or qualitative qualifier. The selection criteria may initially be that the data element is consistently better than other data elements in the plurality of data points in the data set in identifying the biological state class.

“Selecting an initial subset of data elements within each data set” refers to selecting a subset of data elements according to the selection criteria.

“Sharing common data elements” or grammatical equivalents thereof refers to data points sharing common features, e.g., commonly expressed transcripts, proteins, etc.

“Intersection subset” refers to subset of common data elements in a plurality of independent discovery data sets which have been identified independently in each data set as meeting the selection criteria for each independent data set; i.e., in one aspect, a data element in an intersection subset is identified as highly discriminatory (greater than at least 80% specificity and greater than at least about 70% sensitivity in tests to detect or diagnose the biological state class) in each of the independent discovery data sets.

As used herein, “a majority of the initial subsets” refers to greater than 50% of the initial subsets.

The term “measuring” means detecting the presence or absence of marker(s) in the sample, quantifying the amount of marker(s) in the sample, and/or qualifying the type of biomarker. Measuring can be accomplished by methods known in the art and those further described herein, including but not limited to SELDI, immunoassay, and other methods.

“Complementary” in the context of the present invention refers to detection of at least two biomarkers, which when detected together provides increased sensitivity and specificity as compared to detection of one biomarker alone. In certain instances, neither marker by itself have satisfactory discriminatory power, but in combination, are able to discriminate between samples from sources having a state and samples from sources which do not have the state.

The phrase “differentially present” refers to differences in the quantity and/or the frequency of a marker present in a sample taken from patients having a status such as a disease as compared to a control subject. A biomarker is differentially present between two samples if the amount of the biomarker in one sample is statistically significantly different from the amount of the biomarker in the other sample.

“Diagnostic” means identifying the presence or nature of a biological state, such as a pathologic condition, e.g., cancer. Diagnostic methods differ in their sensitivity and specificity. The “sensitivity” of a diagnostic assay is the percentage of samples which test positive for the state (percent of “true positives”). Samples not detected by the assay are “false negatives.” Samples which are not from sources having the biological state and who test negative in the assay, are termed “true negatives.” The “specificity” of a diagnostic assay is 1 minus the false positive rate, where the “false positive” rate is defined as the proportion samples which are from sources which do not have the state which test positive. While a particular diagnostic method may not provide a definitive diagnosis of a biological state, it suffices if the method provides a positive indication that aids in diagnosis. The methods of the present invention preferably provide a specificity of at least 80%, more preferably at least 85%. The methods of the present invention preferably provide a sensitivity of at least 70%, more preferably at least 75%, and most preferably at least 80%.

A “test amount” of a marker refers to an amount of a marker present in a sample being tested. A test amount can be either in absolute amount (e.g., μg/ml) or a relative amount (e.g., relative intensity of signals).

A “diagnostic amount” of a marker refers to an amount of a marker in a sample that is consistent with a diagnosis of a biological state be tested for. A diagnostic amount can be either in absolute amount (e.g., μg/ml) or a relative amount (e.g., relative intensity of signals).

A “control amount” of a marker can be any amount or a range of amounts, which is to be compared against a test amount of a marker. For example, a control amount of a marker can be the amount of a marker in a sample from a source which does not have the biological state (e.g., from a patient who does not have a disease). A control amount can be either in absolute amount (e.g., μg/ml) or a relative amount (e.g., relative intensity of signals).

“Resolve,” “resolution,” or “resolution of marker” refers to the detection of at least one marker in a sample. Resolution includes the detection of a plurality of markers in a sample by separation and subsequent differential detection. Resolution does not require the complete separation of one or more markers from all other biomolecules in a mixture. Rather, any separation that allows the distinction between at least one marker and other biomolecules suffices.

“Detect” refers to identifying the presence, absence or amount of the object to be detected.

As used herein, the term “in communication with” refers to the ability of a system or component of a system to receive input data from another system or component of a system and to provide an output response in response to the input data. “Output” may be in the form of data or may be in the form of an action taken by the system or component of the system.

As used herein, “expression level of a gene product” refers to the amount of a molecule encoded by the gene, e.g., an RNA or polypeptide. The expression level of an mRNA molecule is intended to include the amount of mRNA, which is determined by the transcriptional activity of the gene encoding the mRNA, and the stability of the mRNA, which is determined by the half-life of the mRNA. The gene expression level is also intended to include the amount of a polypeptide corresponding to a given amino acid sequence encoded by a gene. Accordingly, the expression level of a gene can correspond to the amount of mRNA transcribed from the gene, the amount of polypeptide encoded by the gene, or both. Expression levels of a gene product may be further categorized by expression levels of different forms of gene products. For example, RNA molecules encoded by a gene may include differentially expressed splice variants, transcripts having different start or stop sites, and/or other differentially processed forms. Polypeptides encoded by a gene may encompass cleaved and/or modified forms of polypeptides. Polypeptides can be modified by phosphorylation, lipidation, prenylation, sulfation, hydroxylation, acetylation, ribosylation, farnesylation, addition of carbohydrates, and the like. Further, multiple forms of a polypeptide having a given type of modification can exist. For example, a polypeptide may be phosphorylated at multiple sites and express different levels of differentially phosphorylated proteins.

As used herein, a “gene expression profile” refers to a characteristic representation of a gene's expression level in a specimen such as a cell or tissue. The determination of a gene expression profile in a specimen from an individual is representative of the gene expression state of the individual. A gene expression profile reflects the expression of messenger RNA or polypeptide or a form thereof encoded by one or more genes in a cell or tissue. An “expression profile” refers more generally to a profile of biomolecules (nucleic acids, proteins, carbohydrates) which shows different expression patterns among different cells or tissue. The term “expression profile” encompasses the term “gene expression profile”.

As used herein, a “computer program product” refers to the expression of an organized set of instructions in the form of natural or programming language statements that is contained on a physical media of any nature (e.g., written, electronic, magnetic, optical or otherwise) and that may be used with a computer or other automated data processing system of any nature (but preferably based on digital technology). Such programming language statements, when executed by a computer or data processing system, cause the computer or data processing system to act in accordance with the particular content of the statements. Computer program products include without limitation: programs in source and object code and/or test or data libraries embedded in a computer readable medium. Furthermore, the computer program product that enables a computer system or data processing equipment device to act in preselected ways may be provided in a number of forms, including, but not limited to, original source code, assembly code, object code, machine language, encrypted or compressed versions of the foregoing and any and all equivalents.

1. Providing Independent Data Sets

a. Independent Data Sets

The invention provides a data element selection method that reduces the chances of selecting a classifier whose discriminatory power is biased toward sampling differences rather than differences in forms of biological state classes. In particular, the classifier can be a biomarker such as biological molecules exhibiting variability in expression profiling (transcription profiling, proteome profiling, and the like) and clinical sampling. In one preferred aspect of the invention, biomarkers are obtained from proteomic analysis of patient samples. However, the classifier also can be any other phenotypic trait.

Data sets are likely to include biases or preanalytical variables that produce “false” classifiers/biomarkers—that is, biomarkers that differentiate groups not on the basis of the underlying biological state being studied, but the on the basis of the particular bias. For example, if a data set is sex-biased as to the presence/absence of a disease, then certain highly discriminatory classifiers/biomarkers may be differentiating data points based on sex rather than the disease. Similarly, if diseased and normal samples in a data set are handled differently, then a classifier/biomarker may differentiate data points based on differences in handling rather than disease.

In independent data sets the likelihood of the same biases being present is diminished. Therefore, classifiers/biomarkers that are common to all independent data sets are more likely to discriminate based on the biological state of interest, rather than some experimental bias. Accordingly, two data sets are independent if they are collected in such as way as to significantly decrease the chance of being subject to the same bias, i.e., data sets are independent if the populations used to obtain these data sets show a statistically significant difference with respect to at least one preanalytical variable. The best way to diminish biases between data sets is to collect data points from different sites in different geographical locations. In this way, bias factors are more likely to be randomized between the different data sets and, therefore, eliminated in the intersection subset of likely classifiers/biomarkers.

Additional or alternative ways to diminish bias include collecting data points from at different times and/or or from populations which differ as to one or more of such nonlimiting preanalytical variables such as: gender, age, ethnicity, sample collection parameters, sample processing parameters, weight, diet, medication status, medical condition, amount of physical exercise, pregnancy and menstruation, presence and/or level of circulating antibodies, clinical characteristics (e.g., PSA levels, cholesterol levels, familial history of disease, etc.). Preferably, populations differ as to many preanalytical variables.

In the selection of some types of biomarkers (e.g., biomarkers associated with a specific disease), providing populations which differ as to certain preanalytical variables may be particularly important. For example, in identifying biomarkers for decreased protein C levels, providing populations which differ as to other thrombotic risk factors may be desired.

The method starts with a hypothesis that identifying characterizing profiles, such as expression profiles of cells having a given cell state, will lead to the discovery of classifiers, such as biomarkers, which can be used to identify that cell state with high probability (e.g., having specificity of at least about 80% and sensitivity of at least about 70% in diagnostic tests). The expression profiles can be derived from the expression of nucleic acids (e.g., RNA transcripts, including differentially spliced or processed forms thereof), proteins (including modified and/or processed forms thereof), carbohydrates (e.g., lectins) and the like. In one aspect, the cell state reflects the state of a patient from which the cell was derived and is diagnostic of physiological processes being experienced by the patient (e.g., such as pathological responses experienced when the patient has or is developing, or is recovering from a disease).

As a first step, a plurality of independent data sets is obtained. The data sets comprise data points, e.g., a label referring to a sample number or patient number, representing a plurality of samples from multiple sample sources. Each data set comprises a plurality of forms of at least one biological state class, with a plurality of data points (samples) belonging to each of the forms of the class. For example, a biological state class can include, but is not limited to: presence/absense of a disease in the source of the sample (i.e., a patient from whom the sample is obtained); stage of a disease; risk for a disease; likelihood of recurrence of disease; a shared genotype at one or more genetic loci (e.g., a common HLA haplotype; a mutation in a gene; modification of a gene, such as methylation, etc.); exposure to an agent (e.g., such as a toxic substance or a potentially toxic substance, an environmental pollutant, a candidate drug, etc.) or condition (temperature, pH, etc); a demographic characteristic (age, gender, weight; family history; history of preexisting conditions, etc.); resistance to agent, sensitivity to an agent (e.g., responsiveness to a drug) and the like.

Data sets are independent of each other to reduce collection bias in ultimate classifier selection. For example, they can be collected from multiple sources and may be collected at different times and from different locations using different exclusion or inclusion criteria, i.e., the data sets may be relatively heterogeneous when considering characteristics outside of the characteristic defining the biological state class. Factors contributing to heterogeneity include, but are not limited to, biological variability due to sex, age, ethnicity; individual variability due to eating, exercise, sleeping behavior; and sample handling variability due to clinical protocols for blood processing. However, a biological state class may comprise one or more common characteristics (e.g., the sample sources may represent individuals having a disease and the same gender or one or more other common demographic characteristics).

In one aspect, the data sets from multiple sources are generated by collection of samples from the same population of patients at different times and/or under different conditions. However, data sets from multiple sources do not comprise a subset of a larger data set, i.e., data sets from multiple sources are collected independently (e.g., from different sites and/or at different times, and/or under different collection conditions).

In one preferred aspect, a plurality of data sets is obtained from a plurality of different clinical trial sites and each data set comprises a plurality of patient samples obtained at each individual trial site. Sample types include, but are not limited to, blood, serum, plasma, nipple aspirate, urine, tears, saliva, spinal fluid, lymph, cell and/or tissue lysates, laser microdissected tissue or cell samples, embedded cells or tissues (e.g., in paraffin blocks or frozen); fresh or archival samples (e.g., from autopsies). A sample can be derived, for example, from cell or tissue cultures in vitro. Alternatively, a sample can be derived from a living organism or from a population of organisms, such as single-celled organisms.

Thus, for example, in a method for discovering biomarkers for a particular cancer, blood samples for might be collected from subjects selected by independent groups at two different test sites, thereby providing the samples from which the independent data sets will be developed.

b. Collecting Data Points and Generating Data Elements

Data points representing individual samples within a data set are collected. Each data point comprises data elements. A plurality of data points in the data set is characterized by belonging to the same form of biological state class. For example, each data point which belongs to the same biological state class may represent a sample from a patient identified as having a disease of interest for which biomarkers are being identified.

Data elements are features of a data point representing characteristics of the data point. For example, in one aspect, data elements represent expression values of a plurality of different genes in a sample from a patient having a disease shared in common among patients contributing samples to the data set. Each data set comprising data points I through i, will have at least two classes of data points representing at least two forms of a biological state class, present in the sample source providing the data point (class +1) or absent in the sample source providing the data point (class −1). In one aspect, the class −1 data point represents a control (e.g., negative for a disease), though this is not necessarily so. For example, in certain aspects, the class +1 sample represents a certain stage of a disease (e.g., malignant cancer) while class −1 represents another stage of the disease (e.g., benign cells). What the state classes represents will be governed by the nature of the diagnostic test the biomarkers are being selected for.

Preferably, in each data set, the class −1 data points are from sources which do not comprise the at least one common characteristic characterizing a class +1 data points but which are otherwise “matched” with other data points in the data set data set (i.e., collected from the same source, such as a clinical trial site, under similar or the same conditions). Any method for expression profiling known in the art may be used to obtain expression values and is encompassed within the scope of the invention.

Data elements (e.g., gene expression values) can be obtained by transcriptional profiling and/or by proteome profiling. Transcriptional profiling techniques include, but are not limited to: Northern blots, RT-PCR-based differential display methods (Liang and Pardee, Science 257: 967-971, 1992), nuclease protection, representation different analysis (RDA), suppression subtractive hybridization (SSH), and enzymatic degrading subtraction (EDS), gene array profiling (e.g., Affymetrix GeneChip technology), cDNA fingerprinting, subtractive hybridization, serial analysis of gene expression, or SAGE (Lockhar and Winzeler, Nature 405: 827-836, 2000; Velculescu, et al., Science 270: 484-487,1995), and the like. Proteome profiling techniques include, but are not limited to: two-hybrid analysis, fluorescence resonance energy transfer (MET), two dimensional gel electrophoresis, mass spectrometry (e.g., laser desorption/ionization mass spectrometry), fluorescence (e.g. sandwich immunoassay), surface plasmon resonance, ellipsometry and atomic force microscopy.

Other types of biomolecules which are differentially expressed may be profiled to provide data elements. For example, carbohydrates such as lectins (e.g., such as glycans) (see, Sutton-Smith, et al., Biochem. Soc. Symp. 69:105-15, 2002) have diverse expression patterns which can provide data values for data elements comprising a data point.

Preferred methods of expression profiling are high throughput and obtain data elements from greater than about ten, greater than about 50, greater than about 100, greater than about 200, or greater than about 500 samples in data set.

Preferred methods of obtaining data elements include through the use of an array or substrate comprising a plurality of binding partners stably associated therewith (e.g., by attachment, deposition, etc.) for selectively binding to sample components. Such arrays provide probes to detect the presence and/or quantity of multiple different biomolecules (generally, thousands) expressed in a sample in a single assay. Suitable binding partners include, but are not limited to: cationic molecules; anionic molecules; metal chelates; antibodies; single- or double-stranded nucleic acids; proteins, peptides, amino acids; carbohydrates; lipopolysaccharides; sugar amino acid hybrids; molecules from phage display libraries; biotin; avidin; streptavidin; and combinations thereof. Generally, any molecule that has an affinity for desired sample components or which can selectively or specifically absorb a biological molecule can be used as a binding partner. Binding partners stably associated with the array may comprise a single type of molecule or functional group (“monoplex adsorbents”) or can comprise a plurality of different types of molecules or functional groups (“adsorbent species”) to which the marker is exposed (“multiplex adsorbants”). Binding partners or adsorbents can be localized at discrete known locations (i.e., addressable locations) on a probe surface such that a probe surface comprises many different adsorbent species having different binding characteristics. Further, each category of adsorbant may be of the same or different type. For example, nucleic acid molecules adsorbants may comprise a single type of sequence or a plurality of different types of sequences; antibody molecule adsorbants may be monoclonal or polyclonal, and/or may recognize different types of antigens; and such antigens may be from different types of proteins. The substrate material itself may contribute to the selectivity of the array for sample components. Further, different types of eluants or wash solutions can be used to affect or modify adsorption of a sample component to an adsorbent surface and/or to remove unbound materials, for example, by varying pH, ionic strength, hydrophobicity, degree of chaotropism, detergent strength and temperature as is known in the art.

The substrate can be any solid phase onto which a binding partner can be provided. Substrates can be rigid, flexible or semi-flexible, and the shape of the substrate is non-limiting, i.e., substrates can be chips, wafers, tubes, beads, particles, cubes, capillaries, channels, pins, channels, containers, microtiter plates, irregularly shaped surfaces, etc. Substrate materials can include glass, silicon, polymers, etc.

Methods for making and using molecular probe arrays, particularly nucleic acid probe are also disclosed in, for example, U.S. Pat. Nos. 5,143,854; 5,242,974; 5,252,743; 5,324,633; 5,384,261; 5,405,783; 5,409,810; 5,412,087; 5,424,186; 5,429,807; 5,445,934; 5,451,683; 5,482,867; 5,489,678; 5,491,074; 5,510,270; 5,527,681; 5,527,681; 5,541,061; 5,550,215; 5, 554,501; 5,556,752; 5,607,832; 5,658,734; 6,022,963; 6,101,946; 6,150,147; 6,147,205; 6,153,743; 6,140,044. Methods for making and using protein arrays are described in U.S. Pat. Nos. 6,475,809; 6,537,749; 6,475,808; 6,403,309; and 5,770,546, for example. Exemplary carbohydrate arrays (e.g., GlycoChip® glycan chips) are available from Glycominds (Lod 71291, Israel).

Preferably, samples are evaluated after an initial fractionation step to reduce the complexity of the molecules in the sample (i.e., reducing the number of data elements which could characterize a given data point and/or enriching for particular data elements of interest). For example, it can be useful to remove high abundance proteins, such as albumin, from blood before protein analysis. Methods of fractionation include, for example, size exclusion chromatography, ion exchange chromatography, heparin chromatography, affinity chromatography, sequential extraction, gel electrophoresis and liquid chromatography. High performance liquid chromatography (HPLC) also can be used to separate a mixture of biomolecules in a sample based on their different physical properties, such as polarity, charge and size. Methods of fractionation are well known in the art.

The sample can also be fractionated by isolating biomolecules that have a specific characteristic, such as by enriching for sample components having a particular binding affinity for a binding partner. In one aspect, samples are sequentially extracted. In sequential extraction, a sample is exposed to a series of adsorbents to extract different types of biomolecules from a sample. For example, a sample is applied to a first adsorbent to extract certain biomolecules, and an eluant containing non-adsorbent biomolecules (i.e., biomolecules that did not bind to the first adsorbent) is collected. Then, the fraction is exposed to a second adsorbent. This further extracts various biomolecules from the fraction. This second fraction is then exposed to a third adsorbent, and so on.

Samples can also be processed to simplify analysis. For example, nucleic acids can be digested using restriction enzymes as part of a fractionation step to separate nucleic acids comprising particular sequences (restriction enzyme sites) from other sequences. Similarly, proteins can be digested by protease (e.g., such as trypsin), for analysis of peptides (for example, in mass spectroscopy assays).

In one aspect, the substrate comprises a matrix of energy absorbing molecules or “EAMs” that absorbs energy from an ionization source thereby aiding desorption of a sample component, from the surface of the substrate and facilitating analysis of biomolecules adsorbed to the substrate by mass spectroscopy. Suitable EAMS include, but are not limited to: Cinnamic acid derivatives, sinapinic acid (“SPA”), cyano hydroxy cinnamic acid (“CHCA”) and dihydroxybenzoic acid.

In one preferred embodiment, a ProteinChip® Biomarker System (Ciphergen Biosystems, Fremont, Calif.) is used for protein expression profiling of data point samples in a data set and for generating data elements.

In another preferred aspect, one or more sample components are captured on a biochip array and subjected to laser ionization, as in a surface-enhanced laser desorption/ionization time-of-flight (SELDI-TOF) mass spectrometry (MS) assay. In this embodiment, data elements represent data typically obtained SELDI-TOF MS analysis of samples, i.e., the values of data element are the different intensities of signal detected for particular mass/charge ratios (“m/z ratios”) that reflect the molecular weights of the different sample components. These values may be measured against a threshold intensity that is normalized against total ion current. Preferably, logarithmic transformation is used for reducing peak intensity ranges to limit the number of data elements detected.

Other types of mass spectrometry can be used and include the use of any type of apparatus that can measure a parameter which can be translated into mass-to-charge ratios of gas phase ions, i.e., a mass spectrometer. Examples of mass spectrometers are time-of-flight, magnetic sector, quadrupole filter, ion trap, ion cyclotron resonance, electrostatic sector analyzer and hybrids of these. A laser desorption mass spectrometer which uses laser energy as a means to desorb, volatilize, and ionize an analyte also can be used. In one aspect, samples are evaluated by multistage mass spectrometers, such as tandem mass spectrometers. Tandem mass spectrometers are capable of performing two successive stages of m/z-based discrimination or measurement of ions, including of ions in an ion mixture. Analysis may be performed tandem-in-space or tandem-in-time. The phrase thus explicitly includes Qq-TOF mass spectrometers, ion trap mass spectrometers, ion trap-TOF mass spectrometers, TOF-TOF mass spectrometers, Fourier transform ion cyclotron resonance mass spectrometers, electrostatic sector—magnetic sector mass spectrometers, and combinations thereof.

Mass spectral data collected from analysis of probe substrates contacted with samples provide the raw data for the data elements which characterize each data point which is represented by the sample. Preferably, the data elements are pre-processed to eliminate background (e.g., caused by chemical noise from matrix molecules on a SELDI chip) to reduce the number of data elements ultimately evaluated. Background elimination can be performed using a varying width segmented convex hull algorithm as described in Fung and Enderwick, Computational Proteomics Supplement 32: S34-S41, 2002, for example. Peak detection is performed using algorithms known in the art. In one aspect, a peak detection algorithm is used which identifies areas of a mass spectrum as a peak by comparing a given signal to a neighboring valley depth calculation. See, e.g., Fung and Enderwick, supra. Peak intensity is used to represent the relative quantity of a given biomarker expressed in a sample. Signal-to-noise is generally calculated for each peak and used as a filter in further processing. Noise is calculated locally based on the standard deviation from a linear regression of signal around a point of interest.

In a further aspect, peaks of similar molecular weight across all spectra are grouped together into peak clusters while allowing for slight variations in mass. Each cluster represents a different potential biomarker. The peaks used to generate clusters are required to have a minimum signal to noise ratio (e.g., a signal/noise ratio >5 for cluster mass window at 0.3%) and clusters can be selected further according to selected criteria, i.e., such as having having qualified mass peaks within mass/charge (m/z) ratio ranges of between about 1.5 kD-150 kD, and preferably, within about 2 kD-50 kD.

A software program such as an input vector generator can be used to translate data elements obtained from data sets into a binary representation suitable for further analysis.

Preferably, a data element is represented as a vector of numerical values including a value representing the level of a sample component represented by a data element and at least one other characteristic of the sample component/data element, such as its name and/or mass weight.

Thus, for example, the biological state class might be a particular kind of cancer, and the forms of that class might be presence or absence of that cancer. The data points might represents blood samples from individuals who fall into one of the two forms of the class, that is having cancer or cancer free. Data elements are then generated for each data point by analysis of the sample. For example, the samples might be analyzed by gene expression array technology to determine the expression of any number genes. Alternatively, the samples might be analyzed by protein expression profiling, such as SELDI, to determine the expression of any number of proteins, e.g., in the form of mass spectrometry peaks. In each case, each gene or protein is a data element, and the value of each data element is, respectively, the level of expression as measured by the particular technology. The results of this analysis will be two independent data sets populated by the samples in each data set and further characterized by expression levels of the plurality of genes or proteins in each sample. The data might be presented in the form of two data arrays in form of rows and columns: Each array would contain data from a different data set; each row would represent a sample (data point); each column would represent a gene or protein (data element) and each cell would represent the level of expression of the gene or protein (data element value).

2. Qualifying Data Elements

In the next step, data elements obtained from an expression profiling method are qualified using any sort of multivariate analysis. In one method qualification involves using a pattern recognition process, such as a classification model. Classification models can be trained from “known data elements” that are pre-classified (e.g., cancerous or not cancerous). The data elements used to form the classification model can be referred to as a “training data set” or “discovery data set”. Once trained, the classification model can recognize patterns in data derived from data elements from unknown samples. The classification model can then be used to classify the unknown samples into classes. This can be useful, for example, in predicting whether or not a particular biological sample is associated with a certain biological condition (e.g., having a disease or not having a disease).

The discovery data set that is used to form the classification model may comprise raw data or pre-processed data. In some embodiments, raw data can be obtained directly from expression profiling data (e.g., from time-of-flight spectra or mass spectra) and then may be optionally “pre-processed” in any suitable manner. For example, signals above a predetermined signal-to-noise ratio can be selected so that a subset of peaks in a spectrum is selected, rather than selecting all peaks in a spectrum. In another example, a predetermined number of peak “clusters” at a common value (e.g., a particular time-of-flight value or mass-to-charge ratio value) can be used to select peaks. Illustratively, if a peak at a given mass-to-charge ratio is in less than 50% of the mass spectra in a group of mass spectra, then the peak at that mass-to-charge ratio can be omitted from the training data set. Pre-processing steps such as these can be used to reduce the amount of data that is used to train the classification model.

Classification models can be formed using any suitable statistical classification (or “learning”) method that attempts to segregate bodies of data into classes based on objective parameters present in the data. Classification methods may be either supervised or unsupervised. Supervised and unsupervised classification processes are known in the art and reviewed in Jain, IEEE Transactions on Pattern Analysis and Machine Intelligence 22 (1): 4-37, 2000, for example. In selecting a classification method, a balance must be reached between reducing the number of data elements to simplify analysis while minimizing risk of losing useful information.

Unsupervised classification attempts to learn classifications based on similarities in the discovery/training data set, without pre-classifying the data elements (e.g., expression data) from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

In supervised classification, training data containing examples of known categories are presented to a learning mechanism, which learns one more sets of relationships that define each of the known classes using a learning algorithm. New data may then be applied to the learning mechanism, which then classifies the new data using the learned relationships. Differentially expressed sample components (i.e., defining data elements of a data point) may be identified by using a set of data elements whose values represent the expression of the sample components as training data in which the identity (i.e., label corresponding to a sample number/patient number) of each data point is known beforehand. A supervised learning technique derives a classification model (classifier) that assigns data elements obtained from a plurality of data points to a predefined number of known classes with minimum error. The contributions of individual variables to the classification model are then analyzed as a measurement of the value of the data elements, i.e., which data elements are likely to serve as biomarkers with good discriminatory power (i.e., the ability of the biomarker to discriminate between data points which have a biological state from those which do not). Each common data element in each data point, independently for each data set, is qualified based on the ability of the data element to classify a data point into a biological state class, as a function of data element value.

There are different approaches to the derivation of classification models and generally the type of classification approach used is not a limiting feature of the invention.

With traditional statistical approaches, training data is used to estimate the conditional distribution of elements within the data set from data points sharing the at least one characteristic of a biological state being defined (a test class of data elements) and of elements from data points lacking the at least one characteristic (a reference class of data elements). In the traditional statistical approach, training data, whether they are located close to the boundaries between pairs of state classes or far away from the boundaries, contribute equally to the estimation of the conditional distributions from which the final classification model is determined. Since the purpose of classification is to identify accurately the actual boundaries that separate classes of data, training samples close to the separating boundaries should play a more important role than those samples that are far away. Using clinical diagnostic problems as an example, specimens from patients who are borderline cases (e.g., with early stage diseases or benign cases) should be more useful in defining precisely the disease and non-disease classes than those from patients with late stage diseases or young healthy controls.

An example of a statistical approach is discriminant analysis (e.g., Bayesian classifier or Fischer analysis). In Fischer analysis (Fisher, In The Mathematical Theory of Probabilities, Vol. 1, (Macmillan, N.Y.), 1923, or Linear Discriminant Analysis (LDA), the training data from two predefined classes are used to estimate the two class means and to derive a pooled covariance matrix. The means and covariance matrix are then used in determine the classification model. LDA may be preferred where data are conditionally normally distributed and share the same covariance structure.

Other supervised learning techniques include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks and logistic classifiers.

One preferred supervised classification method is a recursive partitioning. Recursive partitioning processes use recursive partitioning trees to classify spectra derived from unknown samples. Some of these methods are described, for example, in WO 01/31579, WO 02/06829, Jan. 24, 2002 and WO 02/42733. Further details about recursive partitioning processes are in U.S. Provisional Patent Application Nos. 60/249,835, filed on Nov. 16, 2000, and 60/254,746, filed on Dec. 11, 2000, and U.S. Non-Provisional patent application Ser. No. 09/999,081, filed Nov. 15, 2001, and Ser. No. 10/084,587, filed on Feb. 25, 2002.

In a particularly preferred embodiment, a supervised learning technique is used which minimizes overfitting, such as a Support Vector Machine (SVM) learning model. See, e.g., Vapnik, In Statistical Learning Theory, (John Wiley & Sons, New York), pp.401-441, 1998. SVM models minimize an empirical risk function that is linked to the classification error of the model over the training data. Using an SVM approach, data elements are characterized by a vector of features (e.g., peptide mass, precursor ion intensity, peptide charge) and used to train an SVM to distinguish between data points sharing the at least one common characteristic and those which do not have the characteristic (for example, to distinguish between data points representing samples from patients having a disease and data points representing samples from patients who do not have the disease). The SVM learning algorithm treats each training sample/data element as a point in higher-dimensional space and searches for a hyperplane that separates positive data elements (associated with the characteristic/disease) and negative data points (not associated with the characteristic/disease) using an optimization algorithm (see, Jaakolla, et al., Proc. Int. Conf. Intell. System. Mol. Biol. 149-58, 1999). The output of optimization is a set of weights, one per data element in the training set. The magnitude of each weight reflects the importance of the data element in defining the separating hyperplane found by optimization, i.e., the likelihood that the data element represents a suitable biomarker.

Data elements with zero weights are correctly classified and far from the hyperplane while those with large weights are incorrectly classified by the hyperplane. The SVM provides a “soft margin” that allows some training data points to fall on the wrong side of a separating hyperplane, charging misclassified data points with a penalty weight. SVM learning techniques are further described in U.S. Pat. No. 6,128,608, for example.

Using an empirical risk minimization approach, such as SVM, the final classification model is largely determined based on training data that are close to biological state class boundaries (i.e., the boundary between the class of data elements from data points sharing the at least one characteristic defining the state and the class of data elements from data points which do not express the at least one characteristic). The solution from SVM, for example, is determined exclusively by a subset of the training samples located along class boundaries (support vectors). The overall data distribution information, as partially represented by the total available training data points, is ignored.

In a preferred aspect, data points are classified by a supervised learning technique which combines SVM with classic linear discrimination analysis in a unified maximum separability analysis (UMSA) procedure. UMSA maximizes the amount of information that may be obtained with a very limited number of samples and is described further in U.S. patent Publication 2003005561.

In one embodiment, a first set of data points in a data set used to define a biological set is selected which represents a class sharing at least one characteristic of the biological state (class +1). A second set of data points is selected which does not have the at least one characteristic defining the biological state (class −1). A modified empirical risk minimization model is derived to obtain an objective function and a plurality of constraints that adequately describe the solution of a classifier to separate the selected samples into the first class and the second class. The model includes terms that individually limit the influence of each sample relative to an importance score (e.g., a value representing how well the data point represents the biological state) of a data point in the solution of the empirical risk minimization model. Solving the modified empirical risk minimization model produces a classifier to separate class +1 data points from class −1 data points.

In one aspect, a data set of m data points xᵢ, where i=1, 2, . . . , m with the corresponding class labels cᵢ, i=1, 2, . . . , m, ε{−1, +1} is defined. Each data is assigned a relative importance score p₁≧0, p₁, representing the trustworthiness of sample xᵢ; minimizing

$$1 / 2 υ \cdot υ + {\sum }_{i = 1}^{m} {p}_{i} {ξ}_{i}$$

subjecting to cᵢ(υ·xᵢ+b)≧1−xᵢ−ξᵢi=1, 2, . . . , m to obtain a solution comprising υ and b, wherein ξᵢ represents a non-negative error for the ith constraint, and constructing an n-dimensional unit vector, d=υ/|υ|=(d₁,d₂. . . dₙ)^{T} from the solution that identifies a direction along which the samples are best separated into a first class labeled as +1 and a second class labeled as −1, respectively, for the set of assigned importance scores p₁, p₂, . . . pₘ.

In another aspect, for a pair of parameters σ and C, a backward stepwise variable selection procedure is performed which includes the steps of (a) assigning each data point in the data set with an initial temporary significance score of zero; (b) computing a temporary significance score for each data point in the data set based on the absolute value of the corresponding element in d=υ/|υ| from the solution and the data point's temporary significance score; (c) finding the data point in the data set with the smallest temporary significance score; (d) assigning the temporary significance score of the data point as its final significance score and removing it from the data set to be used in future iterations; (e) repeating steps (b)-(d) until all data points in the data set have been assigned a final significance score; and (f) constructing vectors s=(s¹, s², . . . sⁿ), wherein s^{k},j=1, . . . , n, represents a computed final significance score for the kth data point of the n data point in the separation of the data points into the first and second classes. The sign, which can be +(Positive) or −(negative),of the kth elements in the n-dimensional unit vector d, sign(dₖ), k=1, 2, . . . , n, indicates whether the corresponding kth data element is up-regulated or down-regulated with respect to the data class labeled as +1, i.e., the data class representing the biological state being defined.

In a further aspect, for a pair of parameters σ and C, a component analysis procedure is performed to determine q unit vectors, q≧min{m, n}, as projection vectors to a q dimensional component space. The component analysis procedure in turn includes the following steps of: (a) setting k=n,; (b) obtaining unit vector d=υ/|υ| from the solution using a current data set; (c) projecting the samples onto a (k−1) dimensional subspace perpendicular to the unit vector d and renaming these projections as the current data set; (d) saving d as a projection vector and setting k=k−1; and (e) repeating steps (b)-(d) until q projection vectors have been determined.

In yet another aspect, a new data point, x=(x₁, x₂, . . . xₙ).^{T} is introduced and a scalar value

$$y = d \cdot x = {\sum }_{i = 1}^{m} {d}_{i} {x}_{j}$$

is computed, The new data point x is assigned to the class +1 if y>y_{c} and to the class corresponding to the label −1 if y≧yc, respectively, where y_{c} is a scalar cutoff value for y.

In a further aspect, a pair of positive values for parameter σ and C is selected, and a positive function Φ(t₁, t₂) that has a range [0, 1], and is monotonically decreasing with respect to its first variable t₁ and monotonically increasing with respect to its second variable t₂, computing δᵢ for each sample xᵢ, i=1, . . . , m, where δᵢ is a quantitative measure of discrepancy between xᵢ's known class membership and information extracted from the data set. A set of assigned importance scores p₁, , p₂, . . . , pₘ, in the form of p₁,=C p₁, (δ_{i,}σ), i=1, . . . , m, and minimizing

$$1 / 2 υ \cdot υ + {\sum }_{i = 1}^{m} {p}_{i} {ξ}_{i}$$

subjecting to cᵢ(υ·xᵢ+b)≧1−xᵢ−ξᵢi=1, 2, . . . , m to obtain a solution comprising υ and b, wherein ξᵢ represents a non-negative error for the ith constraint.

The first class has a class means M₁ and the second class has a class means M₂. δᵢ for each data point xᵢ, i=1, . . . , m, can be set as the shortest distance between the data point xᵢ and the line going through and thereby defined by M₁ and M₂.

The positive function Φ(t1, t2) can take various forms as long as it is monotonically decreasing with respect to its first variable ti and monotonically increasing with respect to its second variable t₂. In one embodiment, a Gaussian function in the form of Φ(δᵢ, σ), exp(−.δ_{i,/}σ²), i=1 . . . , m, is chosen.

Additional data points can be introduced, reiterating the steps above, as described in U.S. patent Publication 20030055615.

If the individual importance scores p₁=p₂=pₘ are the same constant for all training samples, the UMSA classification model becomes the optimal soft-margin hyperplane classification model as commonly used in SVM classification models. The constant C in the term cᵢ(υ·xᵢ+b)≧1−xᵢ−ξᵢ i=1, 2, . . . , m defines the maximum influence any misclassified data point may have on the overall optimization process. The resultant classification model is determined (supported) by only those training samples that are close to the classification boundary and are hence called support vectors.

In the present invention, the UMSA algorithm introduces the concept of relative importance scores that are individualized for each training data point. Through this mechanism, prior knowledge about the individual training data points may be incorporated into the optimization process. The resultant classification model will be preferentially influenced more by the “important” (trustworthy) samples.

Optionally, the individualized importance scores may be computed based on properties estimated from the training samples so that pᵢ=Φ.(xᵢ, D⁺, D⁻)>0. Furthermore, the importance score ₚᵢ may be optionally defined to be inversely related to the level of disagreement of a sample xᵢ to a classifier derived based on distributions of D⁺ and D⁻ estimated from the m training samples. Let this level of disagreement be hᵢ, the following positive decreasing function may be optionally used to compute pᵢ:,

p=Φ(δ)=C−e^{-h}^{ᵢ}²^{_{/s}}², where C>0. where C>1 (equation 2).

In equation 2, the parameter C limits the maximum influence a misclassified training sample may have in the overall optimization process. The parameter s modulates the influence of individual training samples. A very large s will cause equation 2 to be essentially a constant. The UMSA classification model becomes a regular optimal soft-margin hyperplane classification model. On the other hand, a small s amplifies the effect of hᵢ.

As a special case for expression data with very few samples and an extremely large number of variables, which make the direct estimation of conditional distributions difficult, the level of disagreement hi may be optionally defined as the shortest distance between the data point xi and the line that goes through the two class means.

The UMSA derived classification model is both determined by training data points close to the classification boundaries (support vectors) and influenced by additional information from prior knowledge or data distributions estimated from training samples. It is a hybrid of the traditional approach of deriving classification model based on estimated conditional distributions and the pure empirical risk minimization approach. For biological expression data with a small sample size, UMSA's efficient use of information offers an important advantage.

In yet another aspect, the present invention can be utilized to provide following two analytical modules: A) a UMSA component analysis module; and B) a backward stepwise variable selection module, as discussed above and below.

UMSA Component Analysis

The basic algorithm iteratively computes a projection vector d along which two classes of data are optimally separated for a given set of UMSA parameters. The data are then projected onto a subspace perpendicular to d. In the next iteration, UMSA is applied to compute a new projection vector within this subspace. The iteration continues until a desired number of components have been reached. For interactive 3D data visualisation, often only three components are needed. Depending on the shape of data distribution, for many practical problems, three dimensions appear to be sufficient to “extract” all the significant linear separation between two classes of data. The following is a component analysis algorithm for a data set of m samples and n variables:

Inputs:

UMSA parameters C and s;

number of components q≦min(m, n);

data X=(x₁, x₂, . . . , xₘ); and

class labels L=(c₁, C₂, . . . , cₘ),C₁ε{−1,+1}.

Initialization:

component set D←{ };

k←1.

Operation: while k≦q

1. applying UMSA(C, s) on X=(x₁, x₂, . . . , xₘ) and L;

2. dₖ←v/∥v∥; D←D∪{dₖ};

3. xᵢ←xᵢ−(xᵢ^{T}dₖ)dₖ, i=1, 2, . . . , m;

4. k←k+1.

Additionally, the UMSA component analysis method is similar to the commonly used principal component method (PCA) or Singular Value Decomposition (SVD) in that they all reduce data dimension. The difference is that in PCA/SVD, the components represent directions along which the data have maximum variations while in UMSA component analysis, the components correspond to directions along which two predefined classes of data achieve maximum separation. Thus, while PCA/SVD are for data representation, UMSA Component Analysis is for data classification (this is also why in many cases, a three dimensional component space is sufficient for linear classification analysis).

Backward Stepwise Variable Selection Module

For a biological expression data set formulated as an n variables x m samples matrix e, this module implements the following algorithm. The returned vector w contains the computed significance scores of the n variables in separating the two predefined classes of samples:

Inputs:

UMSA parameters C and s;

data e={eⱼᵢ|J=1,2, . . . , n; i=1, 2, . . . , m}; and

class labels L=(c₁, C₂, . . . , cₘ), C₃ε{−1,+1}.

Initialization:

Gₖ←G={gⱼ=(eⱼ₁;eⱼ₂, . . . , eⱼₘ)^{T},j=1, 2, . . . , n};

score vector w=(w¹, w², . . . , wⁿ)^{T}←(0, 0, . . . , 0)^{T}.

Operation: while |Gₖ|>1

1. forming X=(x₁, x₂, . . . , xₘ)←(g₁, g₂, . . . , gₖ)^{T}.

2. applying UMSA(C, s) on X and L,

qₖ←2/∥υ∥ and dₖ←υ/∥υ∥.

3. for all gᵢεGₖ, if qₖ|dₖ^{j|>w}^{j}, w^{j}←qₖ|dₖ^{j}|.

4. Gₖ₋₁←Gₖ−{gᵣ}, where r is determined from

w=min {w}.

gᵢεGₖ

return w.

The training data set and the classification models according to embodiments of the invention can be embodied by computer code that is executed or used by a digital computer. The computer code can be stored on any suitable computer readable media including optical or magnetic disks, sticks, tapes, transmission type media such as digital and analog, etc., and can be written in any suitable computer programming language including C, C++, visual basic, Java, etc.

The output data resulting from training can be displayed on any graphical display interface on a user device connectable to a digital computer or a server to which such a computer is connected (e.g., through the internet). Suitable digital computers include micro, mini, or large computers using any standard or specialized operating system such as a Unix, Windows™ or Linux™ based operating system. The digital computer that is used may be physically separate from the instrument used to obtain values for data elements in a profiling experiment. For example, the computer may be remote from a mass spectrometer that is used to create the spectra of interest, or it may be coupled to the mass spectrometer. The graphical interface also may be remote from the computer, for example, part of a wireless device connectable to the network.

The present invention integrates a re-sampling procedure into the evaluation of expression data to decrease the impact of variation among samples within a data set (e.g., samples from patients from a clinical trial site) and among different data sets (e.g., samples from patients from different clinical trial sites using different exclusion and inclusion criteria and sampling populations with different demographic characteristics). Re-sampling methods such as bootstrapping, bagging, boosting, Monte Carlo simulations, Clest, and the like, are applied, preferably in supervised learning contexts, e.g., using UMSA algorithms as described above.

Accordingly, in one aspect, multiple data sets are independently repeatedly divided into subsets comprising test data points (class +1 data points) and compared to reference or control data points (class −1 data points).

In each re-sampling run, data element(s) are selected that contribute significantly and consistently to the separation of data points having the at least one common characteristic from those which do not, i.e., to identify biomarkers which are diagnostic of the at least one common characteristic. Parameters such as mean, variance and confidence intervals of sampled data elements (e.g., confidence scores for expression data) are measured to determine the distribution of the parameters and to identify outlier scores to form a short list of candidate biomarkers represented by the data elements. For example, expression values (such as mass spectral peaks) with high mean ranks and small standard deviations may be selected to for this list. By performing such analyses independently for each of a plurality of data sets, the possibility of choosing data elements as a result of biases or artifacts in data is reduced, thereby reducing the possibility of false discovery of biomarkers.

By this method, data elements are identified with high confidence values (a selected difference from a null (randomized) distribution being accepted as statistically significant, e.g., p≦0.01) and which are expressed qualitatively in the same manner (overexpressed or underexpressed in both data sets). Outliers of high confidence are ranked from those showing the greatest difference in expression between a test data point and a reference data point (i.e., the most diagnostic) to those which show the least amount of difference (i.e., least diagnostic).

Thus, for example, gene expression or protein expression data from a collection of samples may yield expression data on over one hundred genes or proteins: Each is a data element and its measured expression level is a data element value. After subjecting a data set to the selected from of analysis, the ability of each gene or protein, based on its expression level, to classify a particular sample (data point) as cancerous or non-cancerous (form of biological state class) is determined, or “qualified.” Each gene or protein might then be ranked from most discriminating to least discriminating.

3. Selecting an Initial Subset of Data Elements from Each of the Data Sets

A subset of data elements, e.g., genes or proteins, is now selected from each data set based on selection criteria. Generally, the genes or proteins that are the “best” classifiers from each data set will be selected. For example, the selection criteria might be to “top ten percent” or “the genes or proteins that provide a specified level of sensitivity and/or specificity.” All the data elements from each data set that meet the selection criteria are selected for initial subsets. For example, if there are one hundred genes or proteins that have been ranked in each data set, the top ten percent or discrimators, or ten genes or proteins each, might be selected for the initial data sets.

4. Selecting the Intersection Subset

Most often, these initial subsets will not be identical in terms of the data elements that populate them. However, if they contain data elements in common, these data elements can be selected into an intersection subset. So, for example the initial subset from data set 1 might contain genes or proteins 1, 3, 5, 7 and 9. The initial subset from data set number 2 might contain genes or proteins 1, 2, 3, 4 and 5. The intersection subset could contain any or all of genes or proteins 1, 3 and 5, as the data elements common to both initial subsets.

More specifically, the results from the plurality of data sets are cross-compared to determine a final set of common data elements with consistent expression patterns as a panel of potential biomarkers. Thus, data elements which are selected or qualified as having good “values” or “weights” using the learning algorithms described above in independent discovery data sets are compared, to select an intersection subset of data elements, wherein the data elements in the intersection subset are those which have good values for a plurality of data sets, i.e., the data elements are consistently good biomarkers. Although ideally, a “good value” refers to a data element which has greater than at least 80% specificity and greater than at least about 70% sensitivity in tests to detect or diagnose the biological state class.

5. Testing the Intersection Subset Against an Independent Validation Data Set

At this point, the data elements in the intersection subset are presumptive classifiers or biomarkers. They can be used in multivariate models to generate multivariate classification algorithms.

To construct multivariate predictive models, the data from the plurality of data sets are combined and randomly divided into a discovery training set and a test set. The performance of the panel of potential biomarkers identified from re-sampling and cross-comparison and derived predicted models are evaluated on the test set to identify those biomarkers which survive and which remain highly diagnostic of the at least one common characteristic. Predictive models are validated on independent data elements from one or more new data sets sharing the at least one common characteristic and which have not been involved in biomarker discovery and the model construction process. Independent validation may be performed on data sets which comprise larger populations of data points or with are analyzed using different method (e.g., with a different expression profiling technique from the one used to initially obtain the data elements, such as by an immunoassay), obtaining validation training sets that may be used to identify the most highly discriminatory biomarkers of those being tested. Statistical methods for evaluation of validation data sets included sensitivity and specificity estimation and receiver-operating characteristic (ROC) curve analysis. Such methods are known in the, art.

The multivariate classification algorithm thus generated can be tested against another independent “validation” data set to determine the ultimate power of the algorithm. The validation data set should be independent of all of the discovery data sets used to discover the biomarkers from which the classification algorithm was generated.

Biomarkers can be evaluated after re-sampling, though more preferably, after cross-comparison, to identify additional features of the biomarkers which can be used to characterize validation data sets. For example, sequence information for a peptide or nucleic acid biomarker may be determined. The additional feature(s) may be used to generate probes to test for the presence of the biomarker in test samples (new data points) in data sets used to validate the biomarker. Additional features may include sequence data regarding a larger sequence of which the biomarker sequence is a subsequence (e.g., sequence data for a gene or protein from which the nucleic acid or peptide was derived). Such data may be obtained by using the biomarker sequence to query a database, such as a gene sequence, protein sequence, or glycomic database. Using this method, the sequences of other markers can be identified if these markers are known in the databases.

Preferably, a data element is identified as a biomarker when it is able to predict with greater than 70%, preferably greater than 80%, and still more preferably, greater than 90% accuracy, the presence or absence of a characteristic of a member of a data set. In certain aspects, a plurality of data elements combined can provide the desired predictive value. In certain aspects, combinations with high predictive value may include data elements with lower confidence and may be more predictive than single data elements with higher confidence values. Combinations of data elements suitable for use as biomarkers may be identified by pairing in an ordered or random approach, for example.

Systems for Evaluating Cell States

The invention also includes a computer system that has a database containing features of data elements/biomarkers characteristic of a cell state. In one aspect, the cell state comprises one or more of a stage of differentiation; the expression of a phenotype; a proliferation or stage of a cell cycle; a response to a stimulus, a disease, an agent (e.g., a toxin or a potentially toxic agent, a known or candidate drug; an antibiotic; an infectious or pathological organism; an environmental pollutant, etc), a condition, and the like; environmental pollutant, a candidate drug, etc.) or condition (temperature, pH, etc); and the like. In another aspect, the cell state reflects the status of the source of the cell. For example, the cell state may reflect a disease or other physiological response(s) or conditions being experienced by a patient from which the cell was derived (e.g., such as old age; a psychiatric condition; an addiction; an allergic reaction, etc.).

In one embodiment, the database comprises ranked or clustered biomarkers (i.e., biomarkers divided into subsets based on the discriminatory power of the biomarker). The biomarkers may be ranked or clustered according to association with various parameters. Such parameters include responses to toxins, disease, pollutants, conditions, stressors, developmental stage, drugs, therapeutic agents, antibiotics, and the like. The database comprises biomarkers which show a relatively narrow range of variability in a population for a given cell state but with high discrimination between cell states. For example, the biomarker is reproducibly associated with the parameter (greater than at least 80% specificity and greater than at least about 70% sensitivity in tests to detect or diagnose the parameter) and has a high discriminatory power. However, it should be noted that discriminatory power is not the limiting characteristic of the biomarker. For example, for certain diseases with few or no satisfactory diagnostic tests, a biomarker with lower specificity and/or sensitivity would still have value.

The system additionally comprises a database management system. User requests or queries are formatted in an appropriate language understood by the database management system that processes the query to extract the relevant information from the database of training sets.

The system may additionally include records from an external database or may communicate with such an external database. Examples of external databases include, but are not limited to: GenBank (www.ncbi.nlm.nih.gov/entrez.index.html); KEGG (www.genome.adjp/kegg); SPAD (www.grt.kyushu-u.acjp/spad/index.html); HFUGO (www.gene. ucl.ac.uk/hugo); Swiss-Prot (www.expasy.ch.sprot); Prosite (www. expasy.ch/tools/scnpsitl.html); OMIM (www.ncbi.nlm.nih.gov/omim); GDB (www.gdb.org); and GeneCard (bioinformatics.weizmann.ac.il/cards).

Preferably, the system is connectable to a network to which a network server and one or more clients are connected. The Network may be a local area network (LAN) or a wide area network (WAN), as is known in the art. Preferably, the server includes the hardware necessary for running computer program products (e.g., software) to access database data for processing user requests. For example, one type of user request may be for the system to identify biomarkers associated with a selected cell state. Such as request may provide optional data options, e.g., such as sources of probes that might be used to detect one or more biomarkers (such as a link to a site providing binding partners for the biomarker(s), such as antibodies).

The system also includes an operating system (e.g., UNIX or Linux) for executing instructions from a database management system. In one aspect, the operating system also runs a World Wide Web application, and a World Wide Web server, thereby connecting the server to a network.

Preferably, the system includes one or more user devices that comprises a graphical display interface comprising interface elements such as buttons, pull down menus, scroll bars, fields for entering text, and the like as are routinely found in graphical user interfaces known in the art. Requests entered on a user interface are transmitted to an application program in the system (such as a Web application) for formatting to search for relevant information in one or more of the system databases. Requests or queries entered by a user may be constructed in any suitable database language (e.g., Sybase or Oracle SQL). In one embodiment, a user of user device in the system is able to directly access data using an HTML interface provided by Web browsers and Web server of the system.

The graphical user interface may be generated by a graphical user interface code as part of the operating system and can be used to input data and/or to display inputted data. The result of processed data can be displayed in the interface, printed on a printer in communication with the system, saved in a memory device, and/or transmitted over the network or can be provided in the form of the computer readable medium.

Preferably, the system is in communication with an input device for providing data regarding data elements into the system (e.g., expression values). In one aspect, the input device includes a gene expression profiling system including, e.g., a mass spectrometer, gene chip reader, and the like.

Applications

The invention additionally provides a method of using a computer system comprising identifying the expression level of one or more genes in a tissue or cell sample and comparing the expression level to the expression of a gene included in the training set in the database.

In preferred methods of the present invention, measurements of biomarker(s) in a test sample from a patient are correlated with a status of a patient using a classification algorithm. In one aspect, such measurements are converted into a computer readable form and the system executes an algorithm that classifies the data according to user input parameters. For example, the user may input a query relating to the status (TEST FOR STATUS) which causes the system to test measurements of the biomarker(s)against measurements of the same biomarker in a training set which represents the status (being from data sets of patients having the status). A correspondence between biomarker measurements in the test sample and measurements for the same biomarker(s) in the training set is diagnostic of a high probability (greater than 70%, preferably greater than about 90%, more preferably, greater than about 95%) that the patient has the status.

The methods of the present invention can be performed on any type of patient sample that would be amenable to such methods, e.g., blood, serum and plasma, etc., as described above.

In certain embodiments, a plurality of biomarkers in a sample from the subject are measured, wherein the biomarkers are selected from the group consisting of Marker 1, 2 and Marker n where n≧2. In some methods, the plurality of biomarkers consists of Marker 1, 2 and a Marker 3. The measurement of the plurality of biomarkers can also include measuring a Marker 4. In one aspect, the biomarkers are protein biomarkers and are measured by mass spectroscopy (e.g., such as by SELDI analysis) by immunoassay, or another assay for measuring proteins as is known in the art.

In one aspect of the invention, a method is provided to manage patient treatment based on a determination of the patient's status. For example, if the result of the methods of the present invention is inconclusive or there is reason that confirmation of status is necessary, a health care worker may order more tests. Alternatively, if the status indicates that a medical procedure such as surgery is appropriate, the health care worker may schedule the patient for surgery. Management also may include selection of a treatment regimen, such as drug therapy, chemotherapy, radiotherapy, and the like. Likewise, if the status is negative, e.g., late stage ovarian cancer or if the status is acute, no further action may be warranted. Furthermore, if the results show that treatment has been successful, no further management may be necessary.

Patient management options may be identified by a user of the system or by an expert in communication with the system at a site which is remote from the patient and/or the health care worker or by a combination of the two methods.

The status may be the presence of a disease, risk of developing a disease or risk of reoccurrence of a disease. In one aspect, the disease is cancer (e.g., such as ovarian cancer).

Treatment also may be used to identify additional biomarkers, i.e., physiological responses to treatment may be the least one common characteristic of the biological state class used to obtain and evaluate data sets. Such responses may include a positive response to a treatment, resistance to a treatment, or a negative response to a treatment. By performing the methods described above, biomarkers diagnostic of drug resistance or drug sensitivity may be obtained.

In another aspect, biomarkers from a patient having a particular status are measured over a plurality of time intervals to identify variance in the expression of such biomarkers during such processes as aging, disease, exposure to environmental conditions, stress and the like to identify biomarkers which are consistently diagnostic of the status.

In still another aspect, the invention provides methods for measuring cellular responses to an agent. In one embodiment, measurements of biomarker(s) in a test sample comprising one or more cells are correlated with a cellular response to an agent using a classification algorithm. Such measurements are converted into a computer readable form and the system executes an algorithm that classifies the data according to user input parameters. For example, the user may input a query relating to the status (TEST FOR CELL RESPONSE) which causes the system to test measurements of the biomarker(s)against measurements of the same biomarker in a training set which represents a cell state which is representative of the response (being from data sets of cells having the cell state). A correspondence between biomarker measurements in the test sample and measurements for the same biomarker(s) in the training set is diagnostic of a high probability (greater than 70%, preferably greater than about 90%, more preferably, greater than about 95%) that the cell has the cell state.

In a further aspect, the invention provides methods of screening for therapeutic agents comprising exposing a test sample having a state associated with a pathological condition to a compound and measuring biomarkers to identify the presence of one or more biomarkers correlated with the presence of the state. A compound is identified as a candidate therapeutic agent if the expression of the biomarkers correlated with the state is modulated to more closely resemble the expression of biomarkers correlated with the absence of the state, i.e., the absence of the pathology, in terms of the levels of biomarkers expressed and/or the numbers of biomarkers expressed. Preferably, expression of biomarkers after exposure of the sample to the candidate therapeutic agent is not significantly different from the expression of biomarkers in the absence of the state.

Additional methods for using biomarkers are described in U.S. Provisional Application No. 60/401,837 filed Aug. 6, 2002; U.S. Provisional Application No. 60/441,727 filed Jan. 21, 2003 and Attorney Docket No. 71669/58368-P2 filed Apr. 4, 2003.

Variations, modifications, and other implementations of what is described herein will occur to those of ordinary skill in the art without departing from the spirit and scope of the invention as described and claimed herein and such variations, modifications, and implementations are encompassed within the scope of the invention and the claims recited herein.

All of the references, patents, patent applications, provisional applications and international applications (PCTs) identified herein are expressly incorporated herein by reference in their entireties.

1. A method comprising:
(a) providing at least a first and a second independent discovery data set wherein:
(i) the data sets comprise a plurality of forms of biological state classes;
(ii) each data set comprises a plurality of data points, wherein each data point exhibits one form of a biological state class and each data set comprises a plurality of data points belonging to each of the classes;
(iii) each data point comprises a plurality of data elements, each data element characterized by a value, wherein all data points share a plurality of common data elements; and
(b) qualifying each common data element, independently for each dataset, based on the ability of the data element to classify a data point into a form of biological state class, as a function of data element value;
(c) selecting an initial subset of data elements within each data set, and
(d) selecting an intersection subset of data elements from the initial subsets, wherein each data element in the intersection subset is a member of a majority of the initial subsets.

2. The method of claim 1, wherein the step of selecting the initial subsets comprises using the discovery data sets to train a learning algorithm wherein the learning algorithm ranks the data elements based on a quantitative measure of ability to classify.

3. The method of claim 2, wherein the learning algorithm is a supervised learning algorithm.

4. The method of claim 2, wherein the learning algorithm is an unsupervised learning algorithm.

5. The method of claim 3, wherein the training comprises using support vector machine analysis.

6. The method of claim 2, wherein the training comprises performing linear discrimination analysis.

7. The method of claim 2, wherein, the training comprises performing unified maximum separability analysis (UMSA).

8. The method of claim 1, further comprising independently re-sampling data elements in each data set.

9. The method of claim 1, further comprising, selecting candidate biomarkers from selected data elements and testing one or more of the candidate biomarkers on a validation data set.

10. The method of claim 1, wherein the biological state class comprises a cell state.

11. The method of claim 1, wherein the biological state class is a patient status.

12. The method of claim 1, wherein the biological state class is selected from the group consisting of: presence of a disease; absence of a disease; progression of a disease; risk for a disease; stage of disease; likelihood of recurrence of disease; a genotype; a phenotype; exposure to an agent or condition; a demographic characteristic; resistance to agent, sensitivity to an agent, and combinations thereof.

13. The method of claim 12, wherein the genotype is selected from the group consisting of an HLA haplotype; a mutation in a gene; a modification of a gene, and combinations thereof.

14. The method of claim 12, wherein the agent is selected from the group consisting of a toxic substance, a potentially toxic substance, an environmental pollutant, a candidate drug, and a known drug.

15. The method according to claim 12, wherein the demographic characteristic is selected from the group consisting of: age, gender, weight; family history; and history of preexisting conditions.

16. The method according to claim 12, wherein sensitivity to an agent comprises responsiveness to a drug.

17. The method of claim 9, wherein the one or more candidate biomarkers are diagnostic of the presence of a disease, risk of developing a disease, risk of recurrence of a disease, or stage of the disease.

18. The method of claim 1, wherein values of the data elements in a data point represent levels and/or frequency of components in a data point sample.

19. The method of claim 18, wherein components are selected from the group consisting of: nucleic acids, proteins, polypeptides, peptides, carbohydrates and modified or processed forms thereof.

20. The method of claim 18, wherein levels of components are measured by an expression profiling assay.

21. The method of claim 20, wherein the expression profiling assay comprises measuring the amount and/or form of a nucleic acid.

22. The method of claim 21, wherein expression profiling comprises measuring amplification, mutation, and/or modification of DNA.

23. The method of claim 20, wherein the expression profiling assay comprises measuring the amount and/or form of a protein, polypeptide or peptide.

24. The method of claim 23, wherein the expression profiling assay comprises mass spectrometry.

25. The method of claim 24, wherein the expression profiling assay comprises SELDI analysis.

26. The method of claim 20, wherein the expression profiling assay comprises measuring the amount and/or form of a carbohydrate.

27. The method of claim 1, wherein data elements of data points comprise data relating to the cellular localization of components in a sample.

28. The method of claim 20, wherein expression profiling comprises:
(a) contacting samples with a substrate comprising binding partners for specifically binding to sample components having selected characteristics and
(b) identifying sample components bound to the substrate.

29. The method according to claim 28, wherein binding partners are selected from the group consisting of cationic molecules; anionic molecules; metal chelates; antibodies; single- or double-stranded nucleic acids; proteins, peptides, amino acids; carbohydrates; lipopolysaccharides; sugar amino acid hybrids; molecules from phage display libraries; biotin; avidin; streptavidin; and combinations thereof.

30. The method of claim 28, wherein the binding partners are arrayed on the substrate.

31. The method of claim 2, wherein an assay used to measure levels of data elements in training data sets from which candidate biomarkers are identified is different from an assay used to measure data elements in a validation data set used to validate the candidate biomarker.

32. The method of claim 31, wherein the assay used to measure levels of data elements in training data sets is SELDI.

33. The method of claim 31 or 32, wherein the assay used to measure levels of data elements in validation data sets is an immunoassay.

34. The method of claim 1, wherein the independent discovery data sets are collected from different locations, using different collection protocols, and/or are collected from different populations.

35. The method of claim 1, wherein each discovery data set is from a different clinical trial site.

36. A computer program product comprising a computer readable medium having:
(a) a first computer readable program code providing instructions for causing a computer to input data relating to at least first and second independent discovery data sets wherein:
i) the data sets comprise a plurality of forms of biological state classes;
ii) each data set comprises a plurality of data points, wherein each data point exhibits one form of a biological state class and each data set comprises a plurality of data points belonging to each of the classes; and
iii) each data point comprises a plurality of data elements, each data element characterized by a value, wherein all data points share a plurality of common data elements;
(a) a second computer readable program code providing instructions for qualifying each common data element, independently for each data set, based on the ability of the data element to classify a data point into a biological state class, as a function of data element value and for selecting an initial subset of data elements within each data set, and
(b) a third computer readable program code providing instructions for selecting an intersection subset of data elements from the initial subsets, wherein each data element in the intersection subset is a member of a majority of the initial subsets.

37. The computer program product according to claim 36, wherein selecting the initial subsets comprises using the discovery data sets to train a learning algorithm wherein the learning algorithm ranks the data elements based on a quantitative measure of ability to classify.

38. The computer program product according to claim 37, wherein the learning algorithm is a supervised learning algorithm.

39. The computer program product according to claim 37, wherein the learning algorithm is an unsupervised learning algorithm.

40. The computer program product of claim 37, wherein training comprises support vector machine analysis.

41. The computer, program product of claim 37, wherein training comprises linear discrimination analysis.

42. The computer program product of claim 37, wherein training comprises combining support vector machine analysis and linear discrimination analysis.

43. The computer program product of claim 37, wherein training comprises performing unified maximum separability analysis (UMSA).

44. The computer program product of claim 36, further comprising program code for independently re-sampling data elements in each data set.

45. The computer program product of claim 37, further comprising program code for selecting candidate biomarkers based on ranking by the learning algorithm and for testing one or more of the candidate biomarkers on a validation data set.

46. The computer program product of claim 36, wherein the biological state class comprises a cell state.

47. The computer program product of claim 36, wherein the biological state class comprises a patient status.

48. The computer program product of claim 36, wherein the biological state class is selected from the group consisting of: presence of a disease; absence of a disease; progression of a disease; risk for a disease; stage of disease; likelihood of recurrence of disease; a genotype; a phenotype; exposure to an agent or condition; a demographic characteristic; resistance to agent, sensitivity to an agent, and combinations thereof.

49. The computer program product of claim 48, wherein the genotype is selected from the group consisting of an HLA haplotype; a mutation in a gene; a modification of a gene, and combinations thereof.

50. The computer program product of claim 48, wherein the agent is selected from the group consisting of a toxic substance, a potentially toxic substance, an environmental pollutant, a candidate drug, and a known drug.

51. The computer program product of claim 48, wherein the demographic characteristic is selected from the group consisting of: age, gender, weight; family history; and history of preexisting conditions.

52. The computer program product of claim 48, wherein sensitivity to an agent comprises responsiveness to a drug.

53. The computer program product of claim 45, wherein the one or more candidate biomarkers are diagnostic of the presence of a disease, risk of developing a disease, risk of recurrence of a disease, or stage of the disease.

54. The computer program product of claim 36, wherein values of the data elements in a data point represent levels and/or frequency of components in a data point sample.

55. The computer program product of claim 54, wherein components are selected from the group consisting of: nucleic acids, proteins, polypeptides, peptides, carbohydrates and modified or processed forms thereof.

56. The computer program product of claim 54, wherein levels of components are measured by an expression profiling assay.

57. The computer program product of claim 56, wherein the expression profiling assay comprises measuring the amount and/or form of a nucleic acid.

58. The computer program product of claim 56, wherein expression profiling comprises measuring amplification, mutation, and/or modification of DNA.

59. The computer program product of claim 56, wherein the expression profiling assay comprises measuring the amount and/or form of a protein, polypeptide or peptide.

60. The computer program product of claim 56, wherein the expression profiling assay comprises mass spectrometry.

61. The computer program product of claim 56, wherein the expression profiling assay comprises SELDI analysis.

62. The computer program product of claim 56, wherein the expression profiling assay comprises measuring the amount and/or form of a carbohydrate.

63. The computer program product of claim 36, wherein data elements of data points comprise data relating to the cellular localization of components in a sample.

64. The computer program product of claim 56, wherein expression profiling comprises:
(a) contacting samples with a substrate comprising binding partners for specifically binding to sample components having selected characteristics; and
(b) identifying sample components bound to the substrate.

65. The computer program product of claim 64, wherein binding partners are selected from the group consisting of cationic molecules; anionic molecules; metal chelates; antibodies; single- or double-stranded nucleic acids; proteins, peptides, amino acids; carbohydrates; lipopolysaccharides; sugar amino acid hybrids; molecules from phage display libraries; biotin; avidin; streptavidin; and combinations thereof.

66. The computer program product, wherein an assay used to measure levels of data elements in training data sets from which candidate biomarkers are identified is different from an assay used to measure data elements in a validation data set used to validate the candidate biomarker.

67. The computer program product, wherein the assay used to measure levels of data elements in training data sets is SELDI.

68. The computer program product of claim 66 or 67, wherein the assay used to measure levels of data elements in validation data sets is an immunoassay.

69. The computer program product of claim 36, wherein the independent discovery data sets are collected from different locations, using different collection protocols, and/or are collected from different populations.

70. The computer program product of claim 36, wherein each discovery data set is from a different clinical trial site.

71. A system comprising:
one or more processors for
(a) receiving input data relating to at least first and second independent discovery data sets wherein:
(i) the data sets comprise a plurality of forms of biological state classes;
(ii) each data set comprises a plurality of data points, wherein each data point exhibits one form of a biological state class and each data set comprises a plurality of data points belonging to each of the classes; and
(iii) each data point comprises a plurality of data elements, each data element characterized by a value, wherein all data points share a plurality of common data elements;
(b) executing computer readable program code providing instructions for qualifying each common data element, independently for each data set, based on the ability of the data element to classify a data point into a biological state class, as a function of data element value and for selecting an initial subset of data elements within each data set; and
(c) executing computer readable program code providing instructions for selecting an intersection subset of data elements from the initial subsets, wherein each data element in the intersection subset is a member of a majority of the initial subsets.

72. The system of claim 71, further comprising one or more devices for providing input data to the one or more processors.

73. The system of claim 72, wherein the one or more devices for providing input data comprises a detector for detecting a characteristic of a data element.

74. The system of claim 73, wherein the detector comprises a mass spectrometer.

75. The system of claim 73, wherein the detector comprises a gene chip reader.

76. The system of claim 71, further comprising a memory for storing a data set of ranked data elements.

77. The system of claim 71, further comprising a database of ranked data elements.

78. The system of claim 71, wherein selecting the initial subsets comprises using the discovery data sets to train a learning algorithm wherein the learning algorithm ranks the data elements based on a quantitative measure of ability to classify.

79. The system of claim 78, wherein the learning algorithm is a supervised learning algorithm.

80. The system of claim 78, wherein the learning algorithm is an unsupervised learning algorithm.

81. The system of claim 78, wherein training comprises support vector machine analysis.

82. The system of claim 78, wherein training comprises linear discrimination analysis.

83. The system of claim 78, wherein training comprises combining support vector machine analysis and linear discrimination analysis.

84. The system of claim 78, wherein training comprises performing unified maximum separability analysis (UMSA).

85. The system of claim 71, wherein the system further executes program code for independently re-sampling data elements in each data set.

86. The system of claim 78, wherein the system further executes program code for selecting candidate biomarkers based on ranking by the learning algorithm and for testing one or more of the candidate biomarkers on a validation data set.

87. The system of claim 71, wherein the biological state class comprises a cell state.

88. The system of claim 71, wherein the biological state class comprises a patient status.

89. The system of claim 71, wherein the biological state class is selected from the group consisting of: presence of a disease; absence of a disease; progression of a disease; risk for a disease; stage of disease; likelihood of recurrence of disease; a genotype; a phenotype; exposure to an agent or condition; a demographic characteristic; resistance to agent, sensitivity to an agent, and combinations thereof.

90. The system of claim 89, wherein the genotype is selected from the group consisting of an HLA haplotype; a mutation in a gene; a modification of a gene, and combinations thereof.

91. The system of claim 89, wherein the agent is selected from the group consisting of a toxic substance, a potentially toxic substance, an environmental pollutant, a candidate drug, and a known drug.

92. The system of claim 89, wherein the demographic characteristic is selected from the group consisting of: age, gender, weight; family history; and history of preexisting conditions.

93. The system of claim 89, wherein sensitivity to an agent comprises responsiveness to a drug.

94. The system of claim 86, wherein the one or more candidate biomarkers are diagnostic of the presence of a disease, risk of developing a disease, risk of recurrence of a disease, or stage of the disease.

95. The system of claim 71, wherein values of the data elements in a data point represent levels and/or frequency of components in a data point sample.

96. The system of claim 95, wherein components are selected from the group consisting of: nucleic acids, proteins, polypeptides, peptides, carbohydrates and modified or processed forms thereof.

97. The system of claim 95, wherein levels of components are measured by an expression profiling assay.

98. The system of claim 97, wherein the expression profiling assay comprises measuring the amount and/or form of a nucleic acid.

99. The system of claim 98, wherein expression profiling comprises measuring amplification, mutation, and/or modification of DNA.

100. The system of claim 97, wherein the expression profiling assay comprises measuring the amount and/or form of a protein, polypeptide or peptide.

101. The system of claim 97, wherein the expression profiling assay comprises mass spectrometry.

102. The system of claim 101, wherein the expression profiling assay comprises SELDI analysis.

103. The system of claim 97, wherein the expression profiling assay comprises measuring the amount and/or form of a carbohydrate.

104. The system of claim 71, wherein data elements of data points comprise data relating to the cellular localization of components in a sample.

105. The system of claim 97, wherein expression profiling comprises:
(a) contacting samples with a substrate comprising binding partners for specifically binding to sample components having selected characteristics and
(b) identifying sample components bound to the substrate.

106. The system of claim 105, wherein binding partners are selected from the group consisting of cationic molecules; anionic molecules; metal chelates; antibodies; single- or double-stranded nucleic acids; proteins, peptides, amino acids; carbohydrates; lipopolysaccharides; sugar amino acid hybrids; molecules from phage display libraries; biotin; avidin; streptavidin; and combinations thereof.

107. The system of claim 71, wherein an assay used to measure levels of data elements in training data sets from which candidate biomarkers are identified is different from an assay used to measure data elements in a validation data set used to validate the candidate biomarker.

108. The system of claim 107, wherein the assay used to measure levels of data elements in training data sets is SELDI.

109. The system of claim 107 or 108, wherein the assay used to measure levels of data elements in validation data sets is an immunoassay.

110. The system of claim 71, wherein the independent discovery data sets are collected from different locations, using different collection protocols, and/or are collected from different populations.

111. The system of claim 71, wherein each discovery data set is from a different clinical trial site.
